(19) United States
(12) Patent Application Publication
(10) Pub. No.: US 2007/0197788 A1
(43) Pub. Date: Aug. 23, 2007

(54) METHOD FOR THE PREPARATION OF ENANTIOMER FORMS OF CIS-CONFIGURED 3-HYDROXYCYCLOHEXANE CARBOXYLIC ACID DERIVATIVES USING HYDROLASES

(75) Inventors: Wolfgang Holla, Kelkheim (DE); Stefanie Keil, Hofheim (DE); Christoph Tappertzhofen, Frankfurt (DE)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt (DE)

(21) Appl. No.: 11/669,545

(22) Filed: Jan. 31, 2007

Related U.S. Application Data
(63) Continuation of application No. PCT/EP05/08058, filed on Jul. 23, 2005.

(30) Foreign Application Priority Data
Aug. 7, 2004 (DE) 102004038403.7

(51) Int. Cl. C07F 7/02; C07D 413/02; C07D 263/02

(52) U.S. Cl. 546/14; 546/271.4; 548/215

(57) ABSTRACT

The present invention relates to a process for preparing chiral non-racemic cis-configured cyclohexanols or cyclohexanol derivatives of the formula (I) 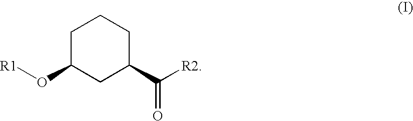 Cis-configured hydroxyl-cyclohexane carboxylic acid derivatives of formula (I) are central building blocks or immediate precursors for the medicinally active compounds which allow a therapeutic modulation of the lipid and/or carbohydrate metabolism and are thus suitable for preventing and/or treating type II diabetes, hyperglycemia and artherosclerosis. The cis-configured hydroxyl-cyclohexane carboxylic acid derivatives of formula (I) are central building blocks or immediate precursors for the medicinally active compounds described in the prior art.

### CROSS-REFERENCE TO RELATED APPLICATIONS

This application is a continuation of International Patent Application No. PCT/EP2005/008058 filed on Jul. 23, 2005 which is incorporated herein by reference in its' entirety which also claims the benefit of priority of German Patent Application No.10/2004 038 403.7 filed on Aug. 7, 2004.

### FIELD OF THE INVENTION

The present invention relates generally to processes for the preparation of compounds and formulations thereof useful in the treatment of metabolic disorders such as hyperlipidemia, diabetes and the consequential cardio-related problems that arise therefrom such as artherosclerosis, serum blood disorders and the like. More specifically, the present invention relates to processes for the preparation of chiral non-racemic cis-configured cyclohexanols or cyclohexanol derivatives which are the central building blocks or immediate precursors for the medicinally active cyclohexane carbonyl aminobutyric acid derivatives and related compounds which allow a therapeutic modulation of lipid and/or carbohydrate metabolism.

### BACKGROUND OF THE INVENTION

The invention relates to a process for preparing chiral non-racemic cis-configured cyclohexanols or cyclohexanol derivatives of the formula (I)

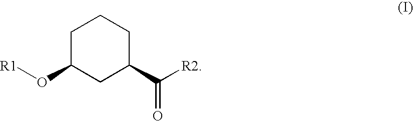

Cis-configured hydroxyl-cyclohexane carboxylic acid derivatives of formula (I) are central building blocks or immediate precursors for the medicinally active compounds described in the DE application No. 103 08 355.3 or WO2004/076426 which allow a therapeutic modulation of the lipid and/or carbohydrate metabolism and are thus suitable for preventing and/or treating type II diabetes and artherosclerosis.

The chemical syntheses of the compounds and derivatives described in the patent applications referred to above, are unsuitable for use as industrial processes: the separation of the isomers and the separation of the enantiomers (racemate resolution) by chromatography on a chiral phase is too complex and much too expensive. Moreover, in order to chromatographically separate the enantiomers, the racemic compound has to be relatively chemically pure, which in many cases can only be achieved by an additional upstream chromatography.

In addition, numerous reactions cannot be carried out on an industrial scale. These include, in particular, alkylations with NaH in DMF, which are known to be a high safety risk (C&EN, Sep. 13, 1982, 5).

The other methods for synthesizing optically pure cis-configured 3-hydroxycyclohexanecarboxylic acid derivatives known in the art are also unsuitable for producing relatively large amounts of the central building blocks of the medicinally active compounds mentioned above or unsuitable for developing an industrial process, since the number of steps required and/or the yield produced thereby, the optical purity thereof and the need for extensive purifications, in particular, cis/trans separations, are unacceptable.

For example: some of the synthetic methods described in the literature for preparing optically pure cis-configured 3-hydroxycyclohexanecarboxylic acid derivatives are based on the hydrogenation or dearomatization of m-hydroxybenzoic acid or its derivatives and the subsequent classical racemate resolution by formation of salts. In the case of the hydrogenation of m-hydroxybenzoic acid in the presence of PtO₂ in ethanol (D. S. Noyce, D. B. Denney, J. Am. Chem. Soc. 1952, 74, 5912; cf. J. A. Hirsch, V. C. Truc, J. Org. Chem. 1986, 51, 2218) it has been described that six crystallizations are required to obtain racemic cis-3-hydroxycyclohexanecarboxylic acid chemically pure in a yield of 13.8%. Noyce and Denney also describe the racemate resolution of cis-3-hydroxycyclohexanecarboxylic acid with the aid of quinine trihydrate. Starting with 500 g of quinine trihydrate and 188.3 g of (R,S)-cis-3-hydroxycyclohexanecarboxylic acid, after several crystallization steps, 162 g of the (+)-cis-3-hydroxycyclohexanecarboxylic acid quinine salt are obtained. Details concerning the optical purity (ee) are not given, only optical rotations are stated. In another method for preparing optically active cis-3-hydroxycyclohexanecarboxylic acid, the precipitation of the racemic cis-configured carboxylic acid with cinchonidine [a) D. S. Noyce, D. B. Denney, J. Am. Chem. Soc. 1952, 74, 5912; b) M. Nakazaki, K. Naemura, S. Nakahara, J. Org. Chem. 1979, 44, 2438] and the subsequent recrystallization of the resulting salt from methanol or ethanol are utilized.

The methods described above are unsuitable however for preparing relatively large amounts of optically active cis-3-hydroxycyclohexanecarboxylic acid derivatives, since the large number of chemical steps and/or purification steps, the use of large amounts of optically pure chiral auxiliaries for racemate resolution, the unavoidable liberation of the desired stereoisomer from the salt and last but not least, the poor total yields that are obtained are unpractical and uneconomical.

The preparation of methyl cis-3-acetoxycyclohexanecarboxylate, albeit in racemic form, has been described in D. S. Noyce, H. I. Weingarten, J. Am. Chem. Soc. 1957, 70, 3098.

A more recent publication (C. Exl, E. Ferstl, H. Honig, R. Rogi-Kohlenprath, Chirality 1995, 7, 211) describes the Rh-catalyzed hydrogenations of (−)-menthyl 3-acetoxybenzoate and (−)-menthyl 4-hydroxybenzoate in methanol/acetic acid at 100 bar and 40° C. and 35° C., respectively. In both reactions, at least four products are formed as well as the desired final product. The substantial disadvantages of the reactions are: a) the large number of required steps owing to the preparation of the optically active benzoic esters and the subsequent removal of the chiral auxiliary; b) technically demanding reaction conditions (100 bar); c) unsatisfactory yields and complicated purifications owing to the large number of byproducts and d) low optical purities. In the end,this process is of only little practical value.

D. A. Evans, G. C. Fu and A. H. Hoveyda (J. Am. Chem. Soc. 1992, 114, 6671) describes the Ir(I)-catalyzed hydroboration of secondary or tertiary amides of 3-cyclohexene carboxylic acid. Although yields and diastereoselection are good, there is the problem of removing the trans-isomer. In addition, the conversion of the amides mentioned into the desired compounds of the formula (I) requires either cleavage of the amide bond under relatively drastic conditions with concomitant partial epimerization and lactonization, or else direct rearrangement of the amides into the desired compounds of formula (I) which means, for example, the stereoselective synthesis of amino acid radicals which entails a large number of steps and is therefore uneconomical. This process is unsuitable for use as an industrial process.

The synthesis of methyl (1R,3R)-3-hydroxy-4-cyclohexenecarboxylate, which is a direct precursor of methyl (1R,3S)-3-hydroxycyclohexanecarboxylate, in a 500 mg scale has been described (J. A. Marshall, S. Xie, J. Org. Chem. 1995, 60, 7230 and literature cited). Key step of the synthesis is an asymmetric [4+2]-cycloaddition between an optically active bisacrylate and butadiene in the presence of TiCl₄. Removal of the chiral auxiliary gives (R)-3-cyclohexenecarboxylic acid with 95% ee which, via iodolactonization and subsequent elimination of HI, is converted into (1R,5R)-7-oxabicyclo[3.2.1]oct-2-en-6-one. Opening of the unsaturated lactone using NaHCO₃/MeOH yields methyl (1R,3R)-3-hydroxy-4-cyclohexenecarboxylate. There have been several descriptions of the synthesis of (R)— or (S)-3-cyclohexene-carboxylic acid via such cyclo-additions. Essentially, the numerous examples differ in the chiral auxiliary used. A few publications may be mentioned by way of example: a) W. Oppolzer, C. Chapuis, D. Dupuis, M. Guo, Helv. Chim. Acta 1985, 68, 2100; b) C. Thom, P. Kocienski, K. Jarowicki, Synthesis 1993, 475; c) B. M. Trost, Y. Kondo, Tetrahedron Lett. 1991, 32, 1613. On an industrial scale, these reactions require particular measures to ensure safe handling of the acrylates and the butadiene.

A great disadvantage of these syntheses are the large amounts of iodine and potassium iodide used for lactonizing the cyclohexene carboxylic acid: in the publication of J. A. Marshall and S. Xie, 1.61 g of iodine (about 1 eq.) and 6.0 g (about 6 eq.) of potassium iodide are required for preparing 460 mg of methyl (1R,3R)-3-hydroxy-4-cyclohexenecarboxylate by intramolecular cyclization of 760 mg of cyclohexenecarboxylic acid. In the publication of A. S. Raw and E. B. Jang, (Tetrahedron 2000, 56, 3285), the iodolactonizations are carried out using as much as three times the amount of iodine. Owing to safety considerations and from an ecological point of view, it is not feasible to carry out such a reaction on a multi-kg scale. In addition, the preparation of the lactones mentioned requires chromatographic purifications.

Phenylseleno- and phenylsulfenollactonizations [a) K. C. Nicolaou, S. P. Seitz, W. J. Sipio, J. F. Blount, J. Am. Chem. Soc. 1979, 101, 3884; b) K. C. Nicolaou, Tetrahedron 1981, 37, 4097] are also not a suitable alternative. Not only are the reagents used and/or the products and byproducts formed highly malodorous, in many cases, they are also toxic and cause ecological damage. In most cases, chromatographic purifications are required to remove unwanted subsequent Se or S byproducts. It is therefore not feasible to carry out such a reaction on a multi-kilogram scale.

The bromolactonization (C. Iwata, A. Tanaka, H. Mizuno, K. Miyashita, Heterocycles 1990, 31, 987 and literature cited therein) also does not provide an alternative for the industrial scale, since bromides or bromine sources are to be avoided for ecological reasons and/or require particular precautions.

(R)-Cyclohexene carboxylic acid can also be further obtained by enzymatic de-symmetrization of 1,2-cyclohex-4-enedicarboxylic esters (P. Kocienski, M. Stocks, D. Donald, M. Perry, Synlett 1990, 38) However, here also the above-mentioned disadvantages of the iodo-lactonization apply.

A further alternative is the reduction of 2-iodo-7-oxabicyclo[3.2.1]octan-6-one (also referred to as 4-iodo-6-oxabicyclo[3.2.1]octan-7-one), the immediate product of the iodolactonization of cyclohexenecarboxylic acid, using Bu₃SnH to give the saturated lactone which can then be converted using, for example, NaOEt in ethanol into ethyl 3-hydroxycyclohexanecarboxylate. The removal of the iodine has been described in numerous examples, inter alia in A. S. Raw, E. B. Jang, Tetrahedron 2000, 56, 3285. Frequently, work-up of the Bu₃SnH reaction and complete removal of the resulting Sn and iodine compounds is difficult and, in many cases, requires additional chromatographic purifications, which is also not desirable for an industrial process. The same applies to the removal of the Se, S and Br compounds in the case of the phenylseleno-, the phenylsulfeno- and the bromolactonization reactions.

The enzymatic racemic resolution of methyl cis-3-hydroxycyclohexanecarboxylate or the tetrahydropyranyl derivative by □-chymotrypsin-catalyzed ester hydrolysis (J. B. Jones, P. W. Marr, Tetrahedron Lett. 1973, 3165) is likewise not a suitable process, since the optical purities of the products are unsatisfactory: The experiments that were carried out gave 42% and 50% ee, respectively. Using an optimized control of the conversion, it is possible to achieve enantiomeric excesses of 85%. On the one hand, this is unsatisfactory, but on the other hand, this is only possible by accepting considerably reduced yields.

The preparation of methyl 3-oxocyclohexene-1-carboxylate and methyl 3-oxocyclohexanecarboxylate by reduction of m-methoxybenzoic acid using sodium in liquid ammonia has been described in M. E. C. Biffin, A. G. Moritz, D. B. Paul, Aust. J. Chem. 1972, 25, 1320.

The stereoselective microbial reduction of racemic 3-oxocyclohexanecarboxylic esters using Rhizopus arrhizus and subsequent separation of the diastereomers also leads to optically active cis-3-hydroxycyclohexanecarboxylic esters (F. Trigalo, D. Buisson, R. Azerad, Tetrahedron Lett., 1988, 29, 6109 and the literature cited). The complicated separation of the diastereomers however, makes a scale-up to the industrial scale unattractive.

The reduction of methyl (R)-3-oxocyclohexanecarboxylate with HLAD (horse liver alcohol dehydrogenase) in the presence of NADH yields the cis/trans mixture of methyl 3-hydroxycyclohexanecarboxylate (J. J. Willaert, G. L. Lemiere, L. A. Joris, J. A. Lepoivre, F. C. Alderweireldt, Bioorganic Chemistry 1988, 16, 223). Accordingly, this method is likewise unsuitable for preparing optically pure cis-3-hydroxycyclohexanecarboxylic acid building blocks.

The asymmetric reduction of isopropyl 3-oxocyclohexene-1-carboxylate with Geotrichum candidum (L. Fonteneau, S. Rosa, D. Buisson, Tetrahydron: Asymmetry 2002, 13, 579), too, is not of any interest, since only the trans-isomer of isopropyl 3-hydroxycyclohexanecarboxylate is formed.

Examination of the reaction of shikimate dehydrogenase with 3-oxocyclohexane-carboxylic acid in the presence of NADPH shows that this enzyme converts the (S)-enantiomer in a yield of 90% into the corresponding trans-3-hydroxycyclo-hexanecarboxylic acid (T. D. H. Bugg, C. Abell, J. R. Coggins, Tetrahedron Lett. 1988, 29, 6779). Accordingly, this reaction is also unsuitable.

Industrial application of the biosynthesis of cyclohexane carboxylic acid in Alicyclobacilus acidocaldarius (formerly Bacillus acidocaldarius) and Streptomyces collinus, which starts with shikimic acid and proceeds via 3-hydroxycyclohexane-carboxylic acid, is not possible, since 3-hydroxycyclohexanecarboxylic acid is the trans-configured (1S,3S)-isomer (B. S. Moore, K. Poralla, H. G. Floss, J. Am. Chem. Soc. 1993, 115, 5267). Accordingly, it is an object of the present invention to develop a process which does not have the disadvantages of the prior art processes described above, all of which however, are hereby incorporated herein by reference for the teachings they do provide.

### SUMMARY OF THE INVENTION

The present invention relates to a process for preparing chiral non-racemic, cis-configured cyclohexanols or cyclohexanol derivatives of the formula (I)

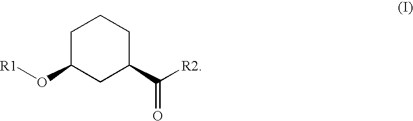

The cis-configured hydroxycyclohexane carboxylic acid derivatives of formula (I) are central building blocks or immediate precursors for the medicinally active compounds described in the DE application No. 103 08 355.3 or WO2004/076426 which allow a therapeutic modulation of lipid and/or carbohydrate metabolism and are thus suitable for preventing and/or treating type 11 diabetes, hyperglycemia and diseases resulting therefrom such as artherosclerosis.

### DETAILED DESCRIPTION OF THE INVENTION

The syntheses, described in the patent applications mentioned above, of the non-racemic, cis-configured cyclohexanol building blocks or immediate precursors thereof for the medicinally active cyclohexane carbonyl aminobutyric acid derivatives and related compounds or derivatives are unsuitable for use as industrial processes. For example, the large number of steps required for the separation of the isomers, the yield produced thereby, the optical purity thereof and the need for extensive purifications, in particular, cis/trans separations, are unacceptable

The present invention provides a process for preparing chiral non-racemic compounds of the formula (Ia) and (Ib) 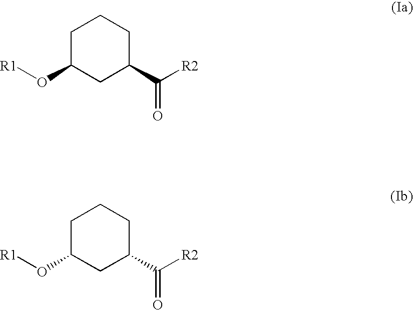
wherein:
R¹ is 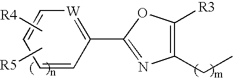
in which:
R3 is selected from the group consisting of H, (C1-C6)-alkyl, (C3-C8)-cycloalkyl, (C1-C3)-alkyl-(C3-C8)-cycloalkyl, phenyl, (C1-C3)-alkyl-phenyl, (C5-C6)-heteroaryl, (C1-C3)-alkyl-(C5-C6)-heteroaryl or (C1-C3)-alkyl which is fully or partially substituted by F;
R4 and R5 are selected from the group consisting of H, F, Cl, Br, CF₃, OCF₃, (C1-C6)-alkyl, O—(C1-C6)-alkyl, SCF₃, SF₅, OCF₂-CHF₂, (C6-C10)-aryl, (C6-C10)-aryloxy, OH, NO₂; and
R4 and R5 together with the ring carbon atoms to which they are attached form a fused partially saturated or unsaturated bicyclic (C6-C10)-aryl or (C5-C11)-heteroaryl ring;

W is CH or N, if n=1;
W is O, S or NR⁶, if n=0;
m is 1-6;
R6 is H, (C1-C6)-alkyl-phenyl, (C1-C6)-alkyl;
or
R1 is an —OH protective group (PG), such as, for example, benzyloxymethyl, benzyl, para-methoxybenzyl, tert-butyldimethylsilyl (TBDMS), tert-butyldiphenylsilyl (TBDPS), tetrahydropyranyl (THP), 1-ethoxyethyl (EE), 1-methyl-1-methoxyethyl or 1-methyl-1-benzyloxyethyl;
and
R2 is 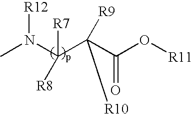
wherein:
p is 0-2;
R7 is selected from the group consisting of H, (C1-C6)-alkyl;
R8 is selected from the group consisting of H, (C1-C6)-alkyl;
R9 is selected from the group consisting of H, F, (C1-C6)-alkyl;
R10 is selected from the group consisting of H, F, (C1-C6)-alkyl, O—(C1-C6)-alkyl, (C2-C6)-alkenyl, (C2-C6)-alkynyl, (C3-C8)-cycloalkyl, phenyl, where alkyl, alkenyl, alkynyl and cycloalkyl may optionally be substituted by one or more radicals from the group consisting of hydroxyl, phenyl, (C5-C11)-heteroaryl, O—(C1-C6)-alkyl and NR13R14, and phenyl may optionally be substituted by one or more radicals from the group consisting of hydroxyl, O—(C1-C6)-alkyl, F and CF₃, with the proviso that R10 is not NR13NR14 or O—(C1-C6)-alkyl, if R9 is F;
R9 and R10 including with the carbon atom that carries them are (C3-C8)-cycloalkyl;
R10 and R12 together are pyrrolidine and piperidine, if n=0;
R11 is selected from the group consisting of H, (C1-C8)-alkyl, benzyl, (C1-C4)-alkyl-(C6-C10)-aryl, (C1-C4)-alkyl-O—(C1-C4)-alkyl, phenyl-(C1-C4)-alkyl, where alkyl, benzyl, phenyl, aryl may optionally be mono- or poly-substituted by O—(C1-C6)-alkyl, OCH₂CH₂—OMe, F, Cl, Br, I, Si(CH₃)₃, OSi(CH₃)₃, Si(iPr)₃, OSi(iPr)₃, OCH₂CH₂—SiMe₃, OCH₂—Si(iPr)₃, O—CH₂—C₆H₅, SO₂C₆H₄-p-Me, SMe, CN, NO₂, CH₂COC₆H₅;
R12 is selected from the group consisting of H, (C1-C6)-alkyl, (C2-C6)-alkenyl, (C2-C6)-alkynyl, benzyl, CO—(C1-C6)-alkyl, CO-phenyl, C(O)—O—(C1-C6)-alkyl, allyloxycarbonyl (ALOC), benzyloxycarbonyl (Cbz, Z), 9-fluorenylmethyloxycarbonyl (FMOC), (C1-C4)-alkyl-(C6-C10)-aryl, (C1-C4)-alkyl-(C5-C11 )-heteroaryl, (C1-C4)-alkyl-O—(C1-C4)-alkyl, phenyl-(C1-C4)-alkyl, (C5-C6)-heteroaryl-(C1-C4)-alkyl; SO₂-(C1-C6)-alkyl, SO₂—(C1-C6)-alkyl-SO₂—(C1-C6)-alkyl, SO₂-phenyl, where phenyl may optionally be substituted by (C1-C6)-alkyl, O—(C1-C6)-alkyl, F, Cl;
R13 is selected from the group consisting of (C1-C6)-alkyl;
R14 is (C1-C6)-alkyl-phenyl, (C1-C6)-alkyl;

wherein

a) Lactone Opening (LO)

Racemic 6-oxabicyclo[3.2.1]octan-7-one of formula (II)

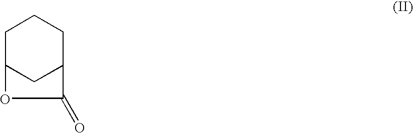

Is reacted with a compound of the formula (III)
HO—R15 (III)
wherein
R15 is selected from the group consisting of H, (C1-C8)-alkyl, (C3-C8)-cycloalkyl, (C2-C8)-alkenyl, (C2-C8)-alkynyl, benzyl, (C1-C4)-alkyl-(C6-C10)-aryl, (C1-C4)-alkyl-(C5-C11 )-heteroaryl, (C1-C4)-alkyl-O—(C1-C4)-alkyl, phenyl-(C1-C4)-alkyl, (C5-C6)-heteroaryl-(C1-C4)-alkyl, where alkyl, benzyl, phenyl, aryl, heteroaryl may optionally be mono- or polysubstituted by phenyl, O—(C1-C6)-alkyl, OCH₂CH₂—OMe, OTs, F, Cl, Br, I, Si(CH₃)₃, OSi(CH₃)₃, Si(iPr)₃, OSi(iPr)₃, OCH₂CH₂—SiMe₃, OCH₂—Si(iPr)₃, OTHP, O—CH₂—C₆H₅, SO₂C₆H₄-p-Me, SMe, CN, NO₂, COOH, CONH₂, CH₂COC₆H₅, CO-benzyloxy, CO—O(C1-C6)-alkyl, NHTs, NHAc, NHBoc, NHAloc, NHbenzyl;

in the presence of suitable bases or acids in a suitable solvent (for example with hydroxides such as NaOH or CsOH in water or, carbonates such as K₂CO₃ in alcohols or, with acids such as HCl in water) or with acid halides such as acetyl chloride in alcohols, preferably acetyl chloride in isopropanol, where in the case of aqueous reactions the work-up conditions determine whether, for example, the Cs (for example if CsOH is used) or the Na (for example if NaOH is used) or the ammonium salt (for example if NH₃ is used) or else the free acid is obtained, to give a racemic cis-configured compound of the formula (IV),

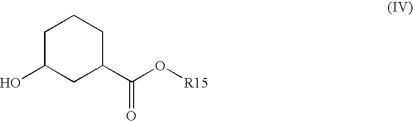

in which R15 is as defined above, or to give a compound of formula (IV) may be present in ionic form as Cs⁺, Li⁺, K⁺, NH₄⁺, Ca²⁺, Ba²⁺, Mg²⁺ salt and in which R15 is also Cs, Li, K, NH₄, Ca, Ba, Mg, and the resulting product is a compound of the formula (IV) where R15=Cs, is further converted with an alkylating agent such as benzyl bromide into a compound of the formula (IV) where R15=CH₂C₆H₅, i.e. another product in which R15 is as defined above.

b) Enzymatic Ester Formation (EF)+Separation(s)

The resulting compounds of the formula (IV) are subjected to a stereoselective enzymatic ester formation (EF), where an acyl donor (such as, for example, a vinyl ester R16-O—CH═CH₂, preferably vinyl acetate, or an acid anhydride R16-O—R16, preferably succinic anhydride and glutaric anhydride) and the enzyme is added to the hydroxyl compounds in an organic solvent such as dichloromethane and the resulting mixture is stirred at from −20 to 80° C. After the reaction has ended, one stereoisomer is present as ester of the formula (Vb) 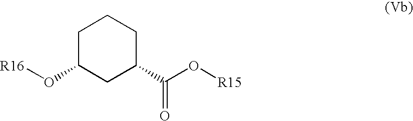
in which
R16 is selected from the group consisting of C(═O)—(C₁-C₁₆)-alkyl, C(═O)—(C₂-C₁₆)-alkenyl, C(═O)—(C₃-C₁₆)-alkynyl, C(═O)—(C₃-C₁₆)-cycloalkyl, where one or more carbon atoms may be replaced by oxygen atoms and may be substituted by 1-3 substituents from the group consisting of F, Cl, Br, CF₃, CN, NO₂, hydroxyl, methoxy, ethoxy, phenyl, CO—O(C₁-C₄)-alkyl and CO—O(C₂-C₄)-alkenyl, where phenyl, CO—O(C₁-C₄)-alkyl and CO—O(C₂-C₄)-alkenyl for their part may be substituted by 1-3 substituents from the group consisting of F, Cl, Br, CF₃, and
R15 is as defined above,

and the other stereoisomer is present unchanged as the alcohol of the formula (IVa)

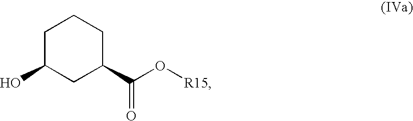

which compounds can, utilizing their different chemical or physicochemical properties (for example R_{f} values or different solubilities in water or other solvents), be separated from one another (separation S), for example by simple chromatography on silica gel, by extraction (for example heptane/methanol or org. solvent/water) or else be processed further by a subsequent chemical follow-up reaction of, for example, the hydroxyl compound, in which the ester does not participate, wherein the enantiomers of the formula (IVa) obtained as alcohol are processed further as described under d), or

c) Ester Cleavage (EC)

The enantiomers of the formula (Vb) obtained as acylated compounds are hydrolyzed by known processes to give chemically enantiomeric alcohols (IVb) or, for example by reaction with K₂CO₃ in methanol, trans-esterified intramolecularly to give the optically active (1S,5R)-6-oxabicyclo[3.2.1]octan-7-one which can be converted into an isomeric form of the product (see schemes below)

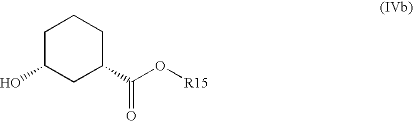

or the compound of the formula (Vb) is converted, for example, by lipase-catalyzed cleavage of both ester functions into the optically active compound of the formula (IVb, where R15=H) which can be converted into an isomeric form of the product (see schemes below);

d) Alkylation (Alk-R1/Alk-PG)

further conversion with compounds of the formula (VI)
R1-X (VI)

in which
R1 is

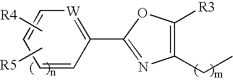

and R3, R4, R5, W, n and m are as defined above, or
R1 is an OH protective group (PG) as defined above, except for THP, EE, 1-methyl-1-methoxyethyl or 1-methyl-1-benzyloxyethyl;
and
X is Cl, Br, I, OTs, OMs, OTf;

in the presence of bases in a suitable solvent to give compounds of the formula (VIIa) or (VIIb);

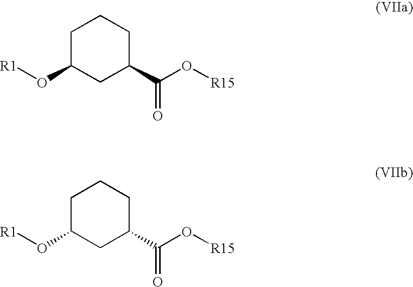

or, in the case of R1=PG, to give compounds of the formula (VIIIa) or (VIIIb)

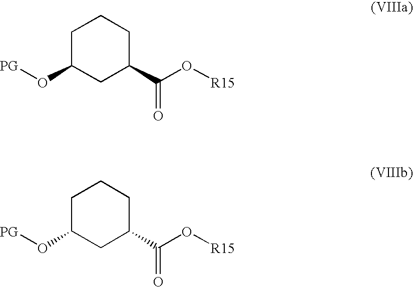

or
R1 is an OH protective group (PG) such as tetrahydropyranyl (THP), 1-ethoxyethyl, 1-methyl-1-methoxyethyl or 1-methyl-1-benzyloxyethyl;

And in order to join the OH protective groups, the compounds of the formula (IVa) or (IVb) are reacted under acid catalysis with the appropriate known enol ethers, also to give compounds of the formula (VIIIa) or (VIIIb);

and

e) Direct Reaction or Ester Cleavage & Coupling (DR or EC+C)

e1) the resulting compounds of the formula (VIIa) or (VIIb) or compounds of the formula (VIIIa) or (VIIIb) are converted in a direct reaction (DR), for example by reacting an amine of the formula (IX)
R2-H (IX)

in which
R2 is

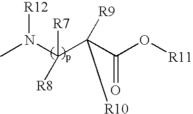

where R7, R8, R9, R10, R11, R12 and p are as defined above,

or the corresponding lithium or dimethylaluminum derivative, or by reacting the compounds of the formula (VIIa) or (VIIb) or the compounds of the formula (VIIIa) or (VIIIb) and the amine or amino acid derivative R²-H of the formula (IX) in the presence of activating reagents or catalysts, such as, for example, the cyanide ion, to give compounds of the formula (Ia) or (Ib) or isomeric forms thereof,

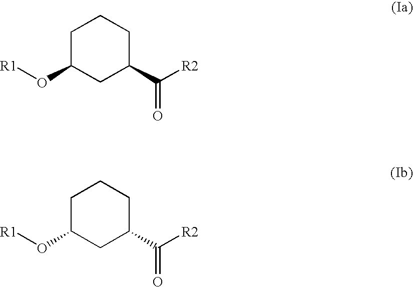

or, in the case of R¹=PG, to give compounds of the formula (Xa) or (Xb),

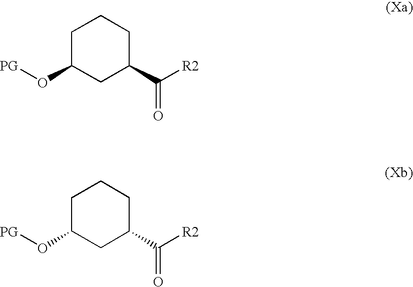

or

e2) the resulting compounds of the formula (VIIa) or (VIIb) or (VIIIa) or (VIIIb) are subjected to an ester cleavage, for example a basic hydrolysis using aqueous NaOH, an acidic hydrolysis using aqueous HCl or an enzymatic hydrolysis using a lipase, or a dehydrogenation using H₂ in the presence of Pd/C, and the resulting compounds of the formula (XIa) or (XIb) or (XIIIa) or (XIIIb)

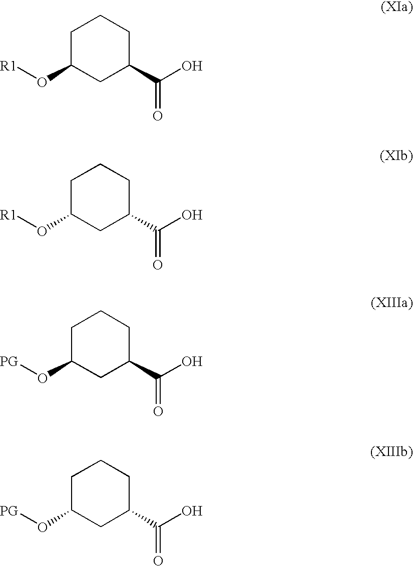

or the corresponding salts, for example the Li, Na, K, C₅ or NH₄ salt of these compounds, to a subsequent coupling with a compound of the formula (IX)
R²—H (IX)

in which
R² is

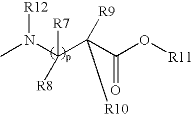

where R7, R8, R9, R10, R11, R12 and p are as defined above,

in the presence of dehydrating or activating reagents, such as, for example, PPA (propane-phosphonic anhydride), TOTU ([cyano(ethoxycarbonyl)methyleneamino]-1,1,3,3-tetramethyluronium tetrafluoroborate), EDC (1-(3-(dimethylamino)propyl)-3-ethylcarbodiimide hydrochloride), HOBt (1-hydroxybenzotriazole), DMAP (4-dimethylaminopyridine), DCC (dicyclo-hexylcarbodiimide), CDI (N,N′-carbonyldiimidazole), to give a compound of the formula (Ia) or (Ib) or an isomeric form thereof;

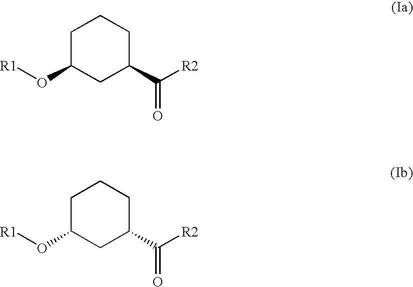

and, if appropriate,

f) Removal of the Protective Group PG (RPG)

if R¹ is an OH protective group (PG) as defined above under R¹, the compounds of the formula (Xa) or (Xb)

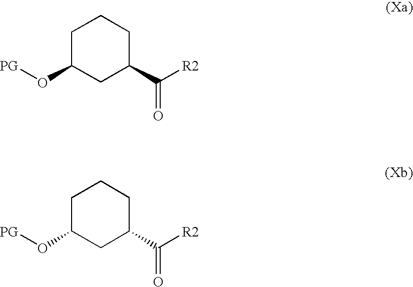

in which R² and PG are as defined above

are converted by removal of the protective group by known processes, such as, for example, removal of PG=benzyloxymethyl or PG=benzyl by hydrogenation over Pd/C, or removal of PG=para-methoxybenzyl using, for example, DDQ (2,3-dichloro-5,6-dicyanobenzoquinone), removal of PG=tert-butyldimethylsilyl, for example, using Bu₄NF, or removal of PG=tetrahydropyranyl (THP), PG=1-ethoxyethyl, PG=1-methyl-1-methoxyethyl or PG=1-methyl-1-benzyloxyethyl, for example, under acid catalysis using p-toluenesulfonic acid or HCl; into compounds of the formula (XIIa) or (XIIb)

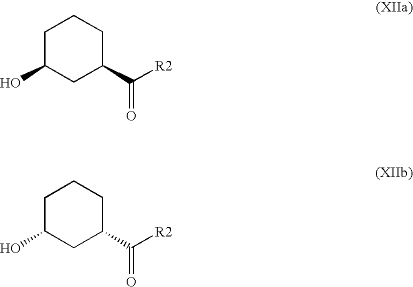

in which R² is as defined above, followed by conversion according to the stated process variants into the compounds of the formula (Ia) or (Ib) or isomeric forms thereof;

it also being possible to change the sequence of the individual reaction steps as described above under A):
A) LO→EF+S[→EC]→Alk-R¹→DR or EC+C→product/isomeric form to:
B) LO→EF+S[→EC]→DR or EC+C→Alk-R¹→product/isomeric form
or
C) LO→DR or EC+C→EF+S→[EC]→Alk-R¹→product/isomeric form
or
D) LO→EF+S→[EC]→Alk-PG→DR or EC+C→RPG→Alk-R¹→product/isomeric form,
or
E) LO→Alk-PG→DR or EC+C→RPG→EF+S[EC]→Alk-R¹→product/isomeric form.

Possible process variants are shown in schemes I to V below:

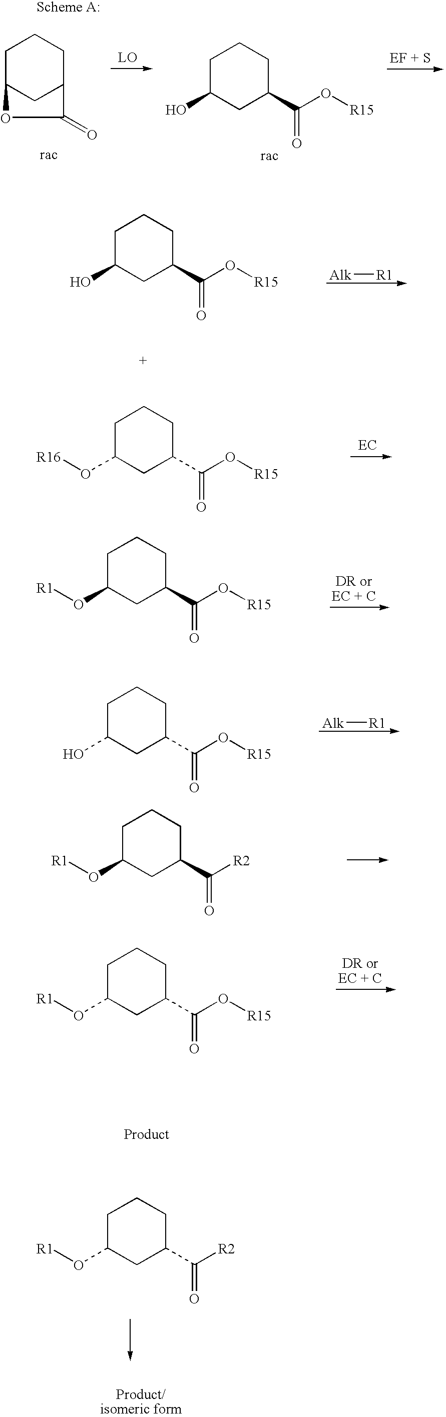

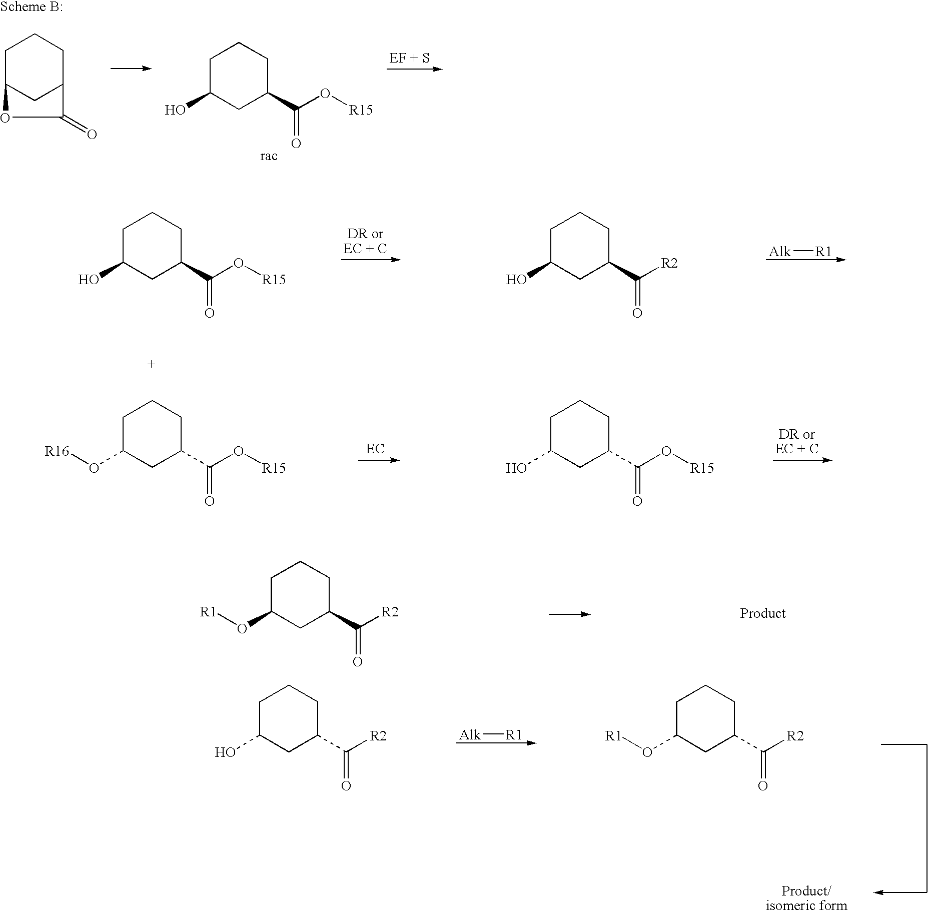

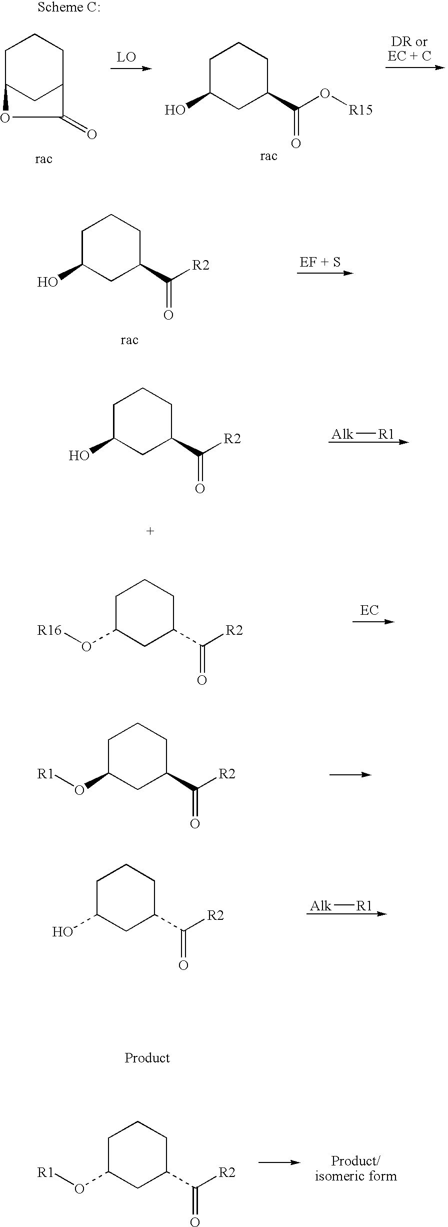

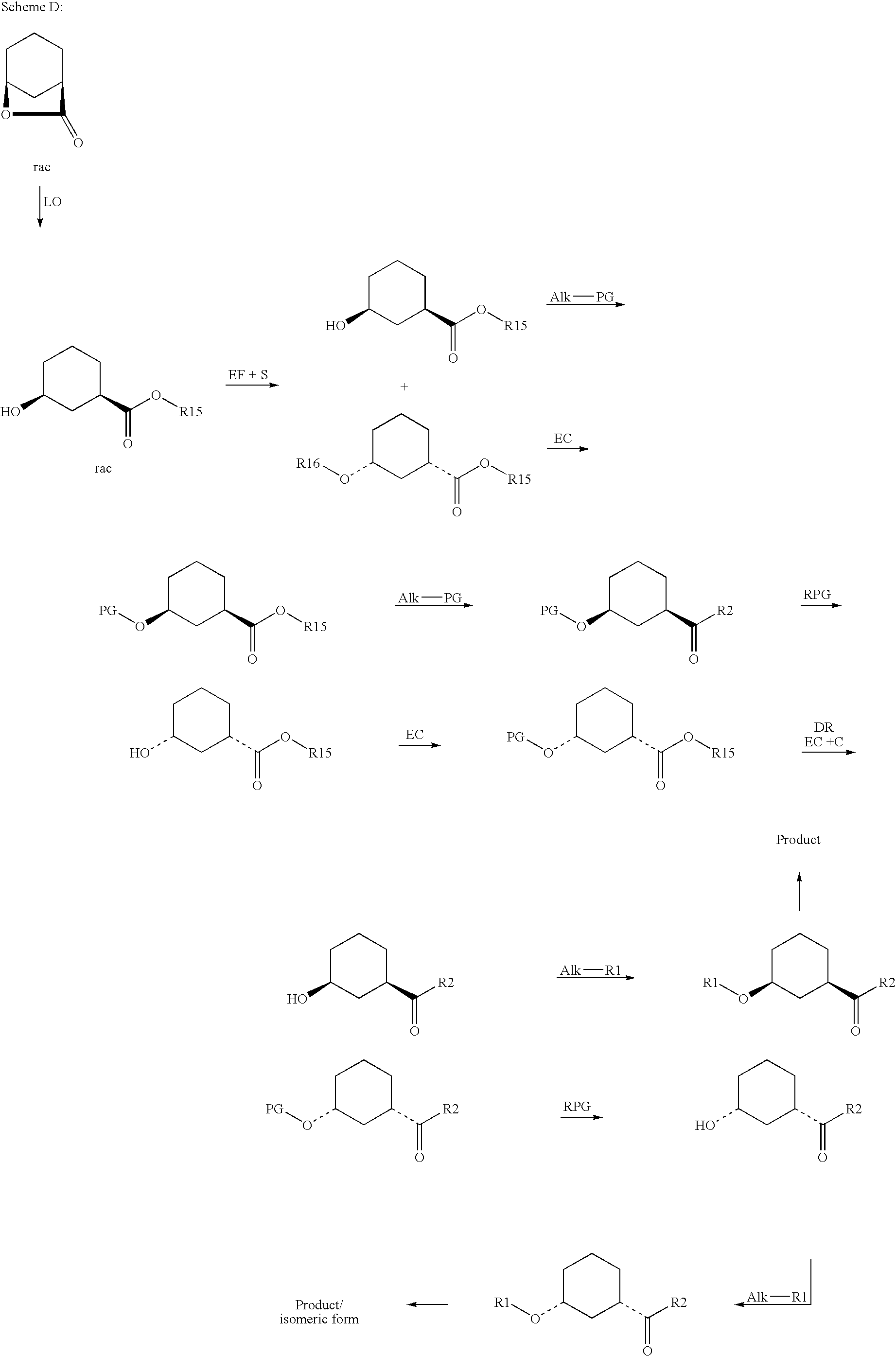

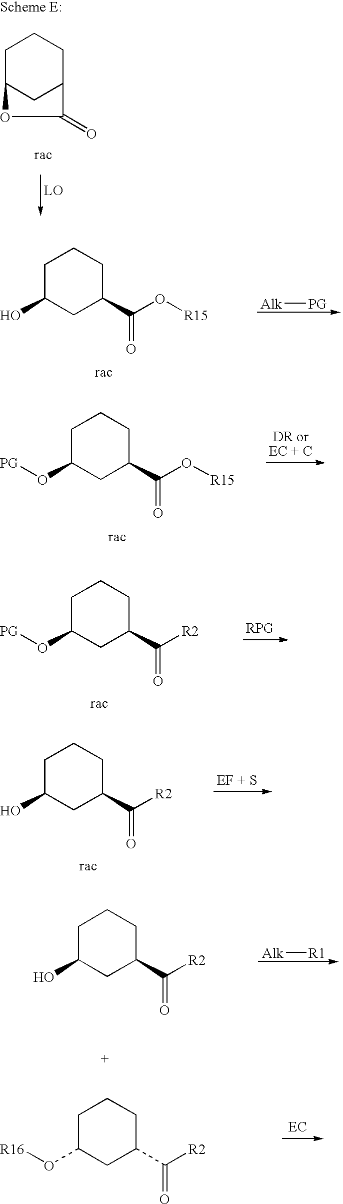

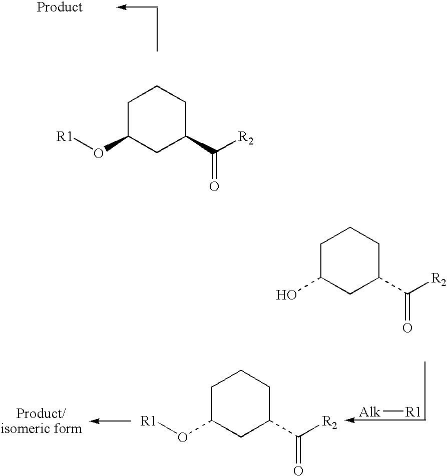

The compounds of the formulae (Ia) and (Ib) have two centers of asymmetry on the cyclohexane ring. Here, the cis attachment is essential. However, it is also possible for further centers of asymmetry to be present, for example in the radical R2. The compounds of the formulae (Ia) and (Ib) can therefore be present in the form of their racemates, racemic mixtures, pure enantiomers, diastereomers and diastereomer mixtures. The present invention embraces all these isomeric forms of the compounds of the formulae (Ia) and (Ib). Even if in some cases this has not been described expressis verbis, these isomeric forms can be obtained by known methods.

A heteroaromatic ring is to be understood as existing as both monocyclic and bicyclic rings having a maximum of 4 heteroatoms, in particular those having up to 4 nitrogen atoms and/or 1 oxygen or 1 sulfur atom, such as, for example: furan, thiophene, thiazole, oxazole, thiadiazole, triazole, pyridine, triazine, quinoline, isoquinoline, indole, benzothiophene, benzofuran, benzotriazole. Aromatic rings may be mono- or bicyclic and also fused, such as, for example, naphthyl, benzo[1,3]dioxole, dihydrobenzo[1,4]dioxin.

The process according to the invention is economical, simple and quick. It does not require equimolar amounts of optically pure starting materials or auxiliaries, nor expensive or hazardous, nor does it require racemate resolution by chromatography on chiral phases, nor unreasonably large amounts of solvents or costly operations.

Preference is given to the abovementioned processes A), B) and D); process A) is particularly preferred.

Preferably, the process for preparing the compounds of the formulae (Ia) and (Ib) is one in which one or more radicals are as defined below:
R¹ is 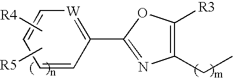
or
R¹ is an OH protective group (PG), such as, for example, benzyloxymethyl, benzyl, para-methoxybenzyl, tert-butyldimethylsilyl (TBDMS), tert-butyldiphenylsilyl (TBDPS), tetrahydropyranyl (THP), 1-ethoxyethyl (EE), 1-methyl-1-methoxyethyl or 1-methyl-1-benzyloxyethyl; and
wherein
R4 is selected from the group consisting of is F, Br, CF₃, OCF₃, (C1-C6)-alkyl, O—(C1-C6)-alkyl, phenyl;
or those in which the substituent R4 is located in the meta or para position;
R5 is hydrogen; or
R4 and R5 together with the phenyl ring=naphthyl; or
R3 is selected from the group consisting of H, (C1-C6)-alkyl, (C3-C8)-cycloalkyl, (C1-C3)-alkyl-(C5-C6)-cycloalkyl, phenyl, (C1-C3)-alkyl-phenyl; or
W is CH, if n=1; or
m is 1; or
R2 is 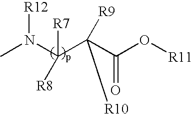
wherein
p is 0; or
R9 is H, (C1-C6)-alkyl; or
R9 and R10 together with the carbon atom that carries them are (C3-C6)-cycloalkyl, in particular cyclopentyl;
R10 is (C1-C6)-alkyl, where alkyl may optionally be substituted by one or more moieties selected from the group consisting of hydroxyl, phenyl, (C5-C11)-heteroaryl, (C1-C6)-alkoxy and NR13R14, where
R13 and R14 are H, (C1-C6)-alkyl;
R11 is H, (C1-C8)-alkyl, benzyl, (C1-C4)-alkyl-(C6-C10)-aryl, where alkyl, benzyl, aryl may optionally be mono- or polysubstituted by OMe, OCH₂CH₂—OMe, F, Cl, Br, Si(CH₃)₃, OSi(CH₃)₃, OCH₂CH₂—SiMe₃, O—CH₂—C₆H₅, SMe, CN, NO₂, CH₂COC₆H₅.

Particular preference is furthermore given to a process for preparing the compounds of the formulae (Ia) and (Ib) in which
R10 is (C1-C4)-alkyl, (C1-C4)-alkyl-O—(C1-C4)-alkyl or benzyl;
R11 is H, (C1-C8)-alkyl, benzyl;
and most preferably,
R10 is (C1-C4)-alkyl or benzyl,
R11 is H, (C1-C8)-alkyl.

A high degree of preference is also given to a process for preparing the compounds of the formulae (Ia) and (Ib)
in which
R4 is selected from the group consisting of Br, CF₃, OCF₃, (C1-C6)-alkyl, O—(C1-C6)-alkyl;
R5 is H, (C1-C6)-alkyl, O—(C1-C6)-alkyl or
R4 and R5 together with the phenyl ring=naphthyl;
R3 is CF₃, (C1-C6)-alkyl, (C3-C6)-cycloalkyl, phenyl;
W is CH, if n=1;
m is 1;
p is 0;
R9 is H, (C1-C6)-alkyl;
R10 is (C1-C6)-alkyl, where alkyl may optionally be substituted by phenyl;
R10 and R12 together with the atoms that carry them are pyrrolidine, if p=0;
R9 and R10 together with the carbon atom that carries them are (C3-C6)-cycloalkyl;
R11 is H;
R12 is H, (C1-C6)-alkyl, benzyl.

The racemic compounds of formula (IV) are prepared by opening 6-oxabicyclo[3.2.1]octan-7-one with alcohols or in the presence of water. Racemic 6-oxabicyclo[3.2.1]octan-7-one (II) is commercially available and can be synthesized, for example, by de-aromatization, for example by hydrogenating m-hydroxybenzoic acid or m-hydroxybenzoic acid derivatives and cyclizing the cis-3-hydroxycyclohexanecarboxyl ic acid.

The opening of the lactone can, as described in the literature for a large number of lactones (for example in M. Carballido, L. Castedo, C. Gonzalez, Tetrahedron Lett. 2001, 42, 3973), be carried out either under acidic or under basic conditions, for example, using water in the presence of hydroxides such as LiOH, for example using water in the presence of acids such as acetic acid, for example using alcohols in the presence of bases such as K₂CO₃ and, for example, using alcohols in the presence of acids such as HCl.

For opening the lactone, it is preferred to use acetyl chloride in alcohols.

To resolve the racemate of the hydroxyl compounds, these are mixed in organic solvents such as, for example, dimethoxyethane (DME), methyl tert-butyl ether (MTBE), diisopropyl ether (DIPE), THF, n-hexane, cyclohexane, toluene, chlorobenzene, acetone, dimethylformamide (DMF), dichloromethane, 1,2-dichloroethane and tert-butanol, acyl donors such as vinyl acetate, vinyl propionate, vinyl butyrate, 2,2,2-trifluoroethyl 2H,2H-perfluorodecanoate, ethoxyvinyl acetate, p-nitro- or p-chlorophenyl acetate, oxime esters, acetic anhydride, propionic anhydride, succinic anhydride, glutaric anhydride, isovaleric anhydride 2,2,2-trichloroethyl butyrate are added and a suitable enzyme is then added to the reaction mixture, which is stirred at from −20 to 80° C. The proportion of cosolvent in the solution is preferably 10-90%; however, if appropriate, it is also advantageous to carry out the enzymatic reaction in a pure acyl donor, for example vinyl acetate, without cosolvent.

After the reaction has ended, the products or the enantiomers can be separated in a simple manner, for example by extraction according to methods known from the literature [a).T. Yamano, F. Kikumoto, S. Yamamoto, K. Miwa, M. Kawada,T. Ito, T. Ikemoto, K. Tomimatsu, Y. Mizuno, Chem. Lett. 2000, 448; b). B. Hungerhoff, H. Sonnenschein, F. Theil, J. Org. Chem. 2002, 67, 1781] all of which are incorporated herein by reference or by using chromatographic methods. In a further method, after completion of the enzymatic reaction, the solubility in water of the remaining hydroxyl compound is increased considerably by derivatization, for example by acylation using cyclic anhydrides, such as glutaric anhydride, or by conversion into a choline ester [a). H. Kunz, M. Buchholz, Chem. Ber. 1979, 112, 2145; b.) M. Schelhaas, S. Glomsda, M. Hänsler, H.-D. Jakubke, H. Waldmann, Angew. Chem. 1996, 108, 82] to achieve separation from the water-insoluble esters or the esters with poor solubility in water by extraction. These articles are also incorporated herein by reference. After the separation, the derivatization of the alcohols can be revised by chemical or enzymatic hydrolysis.

In another separation process of the enantiomers, in the case of the enzymatic acylation, the acyl donor is chosen such that the acylated enantiomer is considerably more water-soluble than the unreacted hydroxyl compound. Suitable acyl donors are, for example, cyclic anhydrides, such as succinic anhydride. After the enzymatic acylation has ended, the acylation product carries a free carboxyl group which allows rapid removal of the product by aqueous extraction in basic media by using saturated aqueous NaHCO₃ solution as an example.

The enzymes used are preferably hydrolases from mammalian livers, such as, for example, lipase from porcine pancreas (Fluka) or from microorganisms, such as, for example, lipase B from Candida antarctica (Roche Diagnostics), lipase A from Candida antarctica (Roche Diagnostics), lipase OF from Candida rugosa (Meito Sangyo), lipase SL from Pseudomonas cepacia (Meito Sangyo), lipase L-10 from Alcaligenes spec. (Roche Diagnostics), lipase QL from Alcaligenes spec. (Meito Sangyo) and glutaryl-7-ACA-acylase (Roche Diagnostics).

Particular preference is given to lipase B from Candida antarctica (Roche Diagnostics), and it may be advantageous to use the free enzyme or an immobilized form of the enzyme, for example one of the three products which are currently commercially available.

Each of the enzymes mentioned can be employed in free or in immobilized form (Immobilized Biocatalysts, W. Hartmeier, Springer Verlag Berlin, 1988). The amount of enzyme is chosen freely depending on the reaction rate or the intended reaction time and on the type of enzyme (for example free or immobilized) and can be determined easily by simple preliminary experiments. The enzyme can be recovered by freeze-drying. Separation (and, if appropriate, later re-use) of the enzyme can be facilitated by immobilization.

By carrying out the reaction in a suitable manner, it is always possible to obtain at least one enantiomer in optically pure form. If the desired product is an optically pure ester, the conversion in the case of the enzymatic ester formation should be below (or equal to) 50%. If the desired product is an optically pure alcohol, in the case of an enzyme-catalyzed ester formation the conversion should be above (or equal to) 50%. The conversion of the enzymatic reaction was determined either by GC or HPLC directly from the reaction mixture or by calculation from the optical purities of the reaction products (ester and acid) which were likewise determined directly from the reaction mixture using GC or HPLC on a chiral phase.

The compounds R1-X of the formula (VI) and the corresponding alcohols R1-OH, which may serve as precursors, are commercially available or can be prepared by methods known from the literature [a) The Chemistry of Heterocyclic Compounds (Ed.: A. Weissberger, E. C. Taylor): Oxazoles (Ed.: I. J. Turchi); b). Methoden der Organischen Chemie [Methods of organic chemistry], Houben-Weyl 4th edition, Hetarene III, subvolume 1; c) O. Diels, D. Riley, Ber. Dtsch. Chem. Ges. 1915, 48, 897; d) W. Dilthey, J. Friedrichsen, J. Prakt. Chem. 1930, 127, 292; e) A. W. Allan, B. H. Walter, J. Chem. Soc. (C) 1986,1397; f) P. M. Weintraub, J. Med. Chem. 1972, 15, 419; g) I. Simit, E. Chindris, Arch. Pharm. 1971, 304, 425; h) Y. Goto, M. Yamazaki, M. Hamana, Chem. Pharm. Bull. 1971, 19 (10), 2050-2057], all of which are incorporated herein by reference.

The compounds R1-X of the formula (VI) are reacted in the presence of bases with the optically pure cis-configured 3-hydroxycyclohexanecarboxylic acid derivatives. Suitable bases are, for example, hydroxides such as KOH, carbonates such as Cs₂CO₃, alkoxides such as KOtBu, and also compounds such as LDA, BuLi, LiHMDS, KHMDS, NaH and NaHMDS. Suitable solvents are, for example, THF, MTBE, DME, NMP, DMF, toluene and chlorobenzene.

Introduction of the OH protective groups (PG) by reacting the compounds of the formulae (IVa) and (IVb) with a compound of the formula (VI) is carried out by methods known from the literature (T. W. Greene, P. G. Wuts, Protective Groups in Organic Synthesis, 3rd edition, John Wiley & Sons, Inc., 1999). all of which are incorporated herein by reference.

In addition, to introduce OH protective groups such as tetrahydropyranyl, 1-ethoxyethyl, 1-methyl-1-methoxyethyl or 1-methyl-1-benzyloxyethyl into the compounds of the formulae (IVa) and (IVb), it is possible to use the corresponding known enol ethers (for example, dihydropyran and ethyl vinyl ether) using methods known from the literature (T. W. Greene, P. G. Wuts, Protective Groups in Organic Synthesis, 3rd edition, John Wiley & Sons, Inc., 1999).

R1 as PG is chosen such that the removal of the PG during the course of the later synthesis can again be facile and selective; PG is thus, for example, selected from the group comprising benzyloxymethyl, benzyl, para-methoxybenzyl, tert-butyldimethylsilyl (TBDMS), tert-butyldiphenylsilyl (TBDPS), 1-ethoxyethyl (EE), or tetrahydropyranyl (THP).

The later removal of the protective group is likewise carried out according to known processes, such as, for example, removal of PG=benzyloxymethyl or PG=benzyl by hydrogenation over Pd/C, or removal of PG=para-methoxybenzyl using, for example, DDQ (2,3-dichloro-5,6-dicyanobenzoquinone), or removal of PG=tert-butyldimethylsilyl or PG=tert-butyldiphenylsilyl using, for example, Bu₄NF, or removal of PG=tetrahydropyranyl (THP), PG=1-ethoxyethyl, PG=1-methyl-1-methoxyethyl or PG=1-methyl-1-benzyloxyethyl, for example under acid catalysis using p-toluenesulfonic acid or HCl.

The amines or amino acid derivatives of the formula (IX) are easily accessible. Both the derivatives of the proteinogenic and the non-proteinogenic amino acids are mainly building blocks known from peptide chemistry, the different isomers of which are commercially available as isomerically pure compounds. In addition, the amino acid derivatives of the formula (IX) used can be prepared with the aid of methods known from the literature [a) Houben-Weyl, Methoden der Organischen Chemie, 4th edition, volume E16d, subvolumes I and II; b) C. Cativiela, M. D. Diaz-de-Villegas, Tetrahedron: Asymmetry 1998, 9, 3517; c) M. Beller, M. Eckert, Angew. Chem. 2000, 112, 1026].

The direct reaction of an ester of the formula (IVa) or (IVb), (VIIa) or (VIIb) or of the formula (VIIIa) or (VIIIb) with an amine or an amino acid derivative of the formula (IX) can be carried out using methods known from the literature [a) M. B. Smith, J. March, March's Advanced Organic Chemistry, 5th edition, John Wiley & Sons, Inc., 2001, p. 510 and literature cited therein; b) literature review R. C. Larock, Comprehensive Organic Transformations, p. 987, VCH Publishers, Inc., 1989], for example via the corresponding lithium or dimethylaluminum derivatives, or in the presence of suitable catalysts, such as, for example, the cyanide ion.

The ester hydrolyses can be carried out according to methods known from the literature (M. B. Smith, J. March, March's Advanced Organic Chemistry, 5th edition, John Wiley & Sons, Inc., 2001, p. 469 and literature cited therein), for example by basic hydrolysis using aqueous NaOH, an acidic hydrolysis using aqueous HCl, or an enzymatic hydrolysis using a lipase or, for example, in the case of a benzyl ester by hydrogenolysis using H₂ in the presence of Pd/C.

The conversion of the cyclohexanecarboxylic acids formed by ester hydrolysis into the compounds of the formula (XIa) or (XIb) is carried out by methods of amide or peptide coupling known from the literature. An abundance of methods for forming amide bonds is available [a) Houben-Weyl, Methoden der Organischen Chemie, 4th edition, volume XV, subvolumes 1 and 2; b) G. Benz in Comprehensive Organic Synthesis (ed.: B. M. Trost), 1991, p. 381; c) Miklos Bodansky, Peptide Chemistry, 2nd edition, Springer Verlag, p. 55]. These as well as all the previously aforementioned scientific articles and journals are incorporated herein by reference.

The examples below serve to illustrate the present invention in more detail. It will be appreciated that every suitable combination of the compounds of the invention with one or more of the aforementioned compounds and optionally one or more other pharmacologically active substances is regarded as falling within the protection conferred by the present invention. The examples detailed below are provided to better describe and more specifically set forth the compounds, processes and methods of this invention. It is to be recognized that they are for illustrative purposes only however, and should not be interpreted as limiting the spirit and scope of the invention as later recited by the claims that follow.

### EXAMPLES

For Scheme A:

### Example 1

### Preparation of Racemic Isopropyl Cis-3-hydroxycyclohexane-1-carboxylate

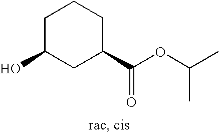

With stirring, 245 ml of acetyl chloride were added slowly to 2.1 l of isopropanol. During the addition, the temperature increased to 45° C. but rapidly fell to 35° C. afterwards. A solution of 350 g (2.72 mol) of racemic 6-oxabicyclo[3.2.1]octan-7-one and 1.4 l of isopropanol was then slowly added dropwise, and the mixture was stirred at 20-25° C. After 3 h and standing overnight, the reaction had ended. The reaction mixture was concentrated under reduced pressure, taken up in about 1.3 l of methylene chloride and washed with 1 l of semisaturated sodium bicarbonate solution. The organic phase was then dried with MgSO₄ and concentrated under reduced pressure; yield: 501 g (98.9%); ¹H-NMR (CDCl₃)|.|||=1.23 (d, 6 H), 1.20-1.45 (m, 4 H), 1.68 (d, 1 H), 1.86 (m, 2 H), 1.95 (m, 1 H), 2.18 (m, 1 H), 2.34 (m, 1 H), 3.63 (m, 1 H), 5.00 (sept, 1 H).

### Example 2

### Enzymatic Racemate Resolution of Isopropyl Cis-3-hydroxycyclohexane-1-carboxylate

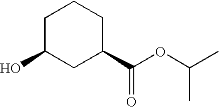

800 g of racemic isopropyl 3-hydroxycyclohexane-1-carboxylate were slowly stirred with 1.5 l of vinyl acetate, 5 l of methylene chloride and 137 g of Novozyme 435 at 20-23° C. After about 4 h, the mixture was filtered off and concentrated under reduced pressure. This gave 940 g which were chromatographed on 6 kg of silica gel (n-heptane/EA 2:1—EA/n-heptane 3:1): 1. Fraction, 484 g, isopropyl (1S,3R)-3-acetoxycyclohexane-1-carboxylate; ¹H-NMR (CDCl₃): □=1.22 (d, 6 H), 1.2-1.6 (m, 4 H), 1.8-2.0 (m, 3 H), 2.03 (s, 3 H), 2.20 (m, 1 H), 2.36 (m, 1 H), 4.70 (m, 1 H), 5.00 (sept, 1 H); 80% ee (HPLC on Chiralpak ADH 32 250×4.6; 1 ml/min, heptane/EtOH 3:1). 2. Mixed fraction. 3. Fraction, 324 g of isopropyl (1R,3S)-3-hydroxycyclohexane-1-carboxylate; ¹H-NMR (CDCl₃): □=1.23 (d, 6 H), 1.20-1.45 (m, 4 H), 1.68 (d, 1 H), 1.86 (m, 2 H), 1.95 (m, 1 H), 2.18 (m, 1 H), 2.34 (m, 1 H), 3.63 (m, 1 H), 5.00 (sept, 1 H); >99% ee (HPLC on Chiralpak ADH 32 250×4.6; 1 ml/min, heptane/EtOH 3:1).

### Example 3

### Preparation of Isopropyl (1R,3S)-3-(5-methyl-2-p-tolyloxazol-4-ylmethoxy)-Cyclohexane-carboxylate by Alkylation of Isopropyl (1R,3S)-3-hydroxycyclohexane-1-carboxylate using 4-iodomethyl-5-methyl-2-p-tolyloxazole

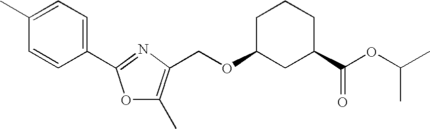

Under N₂, 100 g (0.54 mol) of isopropyl (1R,3S)-3-hydroxycyclohexane-1-carboxylate and 151 g (0.48 mol) of 4-iodomethyl-5-methyl-2-p-tolyloxazole were initially charged in 1 l of NMP and cooled to −20° C. Over a period of about 1 h, 20.4 g of NaOH were added a little at a time. During the addition, the temperature was kept below −15° C. The mixture was then stirred at −15° C. After 7 h, the reaction had ended. The reaction mixture was poured into a mixture of 3 l of water and 40 ml of glacial acetic acid. The product was extracted with MTB ether (2×700 ml). The organic phase was concentrated under reduced pressure which gave 180 g of crude product which was directly reacted further as described in example 4 et seq.

### Example 4

### Preparation of (1R,3S)-3-(5-methyl-2-p-tolyloxazol-4-ylmethoxy)cyclohexane-carboxylic Acid by Hydrolysis of the Isopropyl Ester

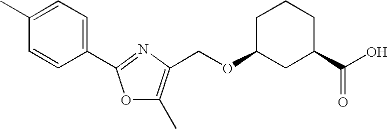

About 360 g from two runs of example 3 were dissolved in 1.6 l of NMP, 400 ml of NaOH were added and the mixture was stirred at RT. After about 1.5 h the reaction solution was poured into 6 l of water and washed three times with in each case 2 l of MTB ether, and using about 450 ml of conc. HCl, the aqueous phase was adjusted to pH 1. The product, which precipitated during this operation, was filtered off, washed with water and dried at 50° C.; yield: 85 g; m.p. 144-146° C.; ¹H-NMR (CDCl₃): □=1.23-1.53 (m, 4 H), 1.85-2.1 (m, 3 H), 2.36-2.45 (m, 8 H), 3.45 (m, 1 H), 4.49 (dd, 2 H), 7.23 (d, 2 H), 7.88 (d, 2 H).

### Example 5

### Preparation of tert-butyl 3-methyl-2(S)-{[(1R,3S)-3-(5-methyl-2-p-tolyloxazol-4-ylmethoxy)cyclohexane-carbonyl]amino}butyrate by coupling of (1R,3S)-3-(5-methyl-2-p-tolyloxazol-4-yl methoxy)cyclohexane-carboxylic acid with t-butyl (S)-valinate hydrochloride

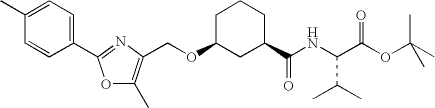

With stirring, 85 g of (1R,3S)-3-(5-methyl-2-p-tolyloxazol-4-ylmethoxy)cyclohexane-carboxylic acid, 70.4 g of t-butyl (S)-valinate hydrochloride and 128 ml of triethylamine were initially charged in 1.39 l of DMF. The mixture was stirred for about 10 min and then cooled to 0° C. (ice/methanol), and 101 g of TOTU were added slowly, a little at a time. The mixture was stirred at 0° C. for 15 min and then at about 20° C. After 2 h, the reaction had ended. The entire reaction mixture was poured into 4.5 l of water and extracted 3× with in each case 700 ml of MTB ether, and the combined organic phases were washed with about 1 l of water to remove residual DMF, dried with MgSO₄ and concentrated under reduced pressure. The residue was triturated with DIPE and filtered off with suction; yield: 82 g. The mother liquor was concentrated and the residue was once more triturated with DIPE; total yield: 90 g; ¹H-NMR (CDCl₃): □=0.87-0.92 (2 d, 6 H), 1.25-1.55 (m, 4 H), 1.46 (s, 9 H), 1.88 (m, 2 H), 2.10-2.35 (m, 4 H), 2.38 (s, 3 H), 2.40 (s, 3 H), 3.47 (m, 1 H), 4.46 (dd, 1 H), 4.49 (s, 2 H), 5.97 (d, 1 H), 7.22 (d, 2 H), 7.88 (d, 2 H).

### Example 6

### Preparation of 3-methyl-2(S)-{[(1R,3S)-3-(5-methyl-2-p-tolyloxazol-4-ylmethoxy)-cyclohexanecarbonyl]amino}butyric acid

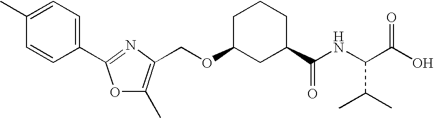

141.0 g of tert-butyl 3-methyl-2(S)-{[(1R,3S)-3-(5-methyl-2-p-tolyloxazol-4-ylmethoxy)cyclohexanecarbonyl]amino}butyrate were dissolved in 700 ml of methylene chloride, and 252 ml of trifluoroacetic acid were added. The reaction mixture was heated at reflux. After a reaction time of about 10 hours, the reaction solution was concentrated under reduced pressure and, twice, about 100 ml of toluene were added and the mixture was concentrated under reduced pressure. The resulting residue was taken up in 1 l of water, and about 150 ml of 33% strength aqueous NaOH were added. The Na salt of the carboxylic acid was dissolved, and the solution was washed twice with in each case 70 ml of MTB ether. Using conc. HCl, the aqueous phase was then adjusted to pH 1. The desired product precipitated and was filtered off with suction, washed with water, dried at 50° C. under reduced pressure and digested with about 1 1 of ethyl acetate; yield: 118.8 g; m.p. 195-196° C.; ¹H-NMR (DMSO): □=0.86 (d, 6 H), 1.05-1.3 (m, 4 H), 1.64 (m, 1 H), 1.76 (m, 1 H), 2.04 (m, 4 H), 2.36 (s, 3 H), 2.38 (s, 3 H), ˜3.35 (m, 1 H), 4.13 (dd, 1 H), 4.40 (s, 2 H), 7.31 (d, 2 H), 7.81 (d, 2 H), 7.84 (d, 1 H), 12.5 (s, br., 1 H).

### Example 7

### 3-Methyl-2(S)-{[(1R,3S)-3-(5-methyl-2-p-tolyloxazol-4-ylmethoxy)cyclohexane-carbonyl]amino}pentanoic acid

Analogously to the reaction sequence examples 2-6, isopropyl (1R,3S)-3-hydroxy-cyclohexane-1-carboxylate, 4-iodomethyl-5-methyl-2-p-tolyloxazole and tert-butyl (S)-leucinate hydrochloride gave the product 3-methyl-2(S)-{[(1R,3S)-3-(5-methyl-2-p-tolyloxazol-4-ylmethoxy)cyclohexanecarbonyl]amino}pentanoic acid; C₂₅H₃₄N₂O₅ (442.56), MS (ESI): 443 (M+H⁺).

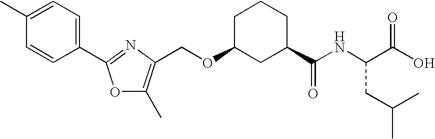

### Example 8

### 3-Methyl-2(S)-{[(1R,3S)-3-(5-methyl-2-p-tolyloxazol-4-ylmethoxy)cyclohexane-carbonyl]amino}propionic acid

Analogously to example 7, isopropyl (1R,3S)-3-hydroxycyclohexane-1-carboxylate, 4-iodomethyl-5-methyl-2-p-tolyloxazole and tert-butyl (S)-alaninate hydrochloride gave the product 3-methyl-2(S)-{[(1R,3S)-3-(5-methyl-2-p-tolyloxazol-4-ylmethoxy)-cyclohexanecarbonyl]amino}propionic acid; C₂₂H₂₈N₂O₅ (400.48), MS (ESI): 401 (M+H⁺).

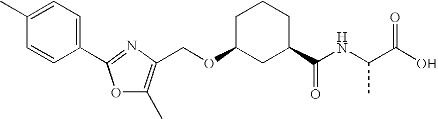

For scheme B:

### Example 9

### Preparation of isopropyl (1R,3S)-3-hydroxycyclohexane-1-carboxylate by enzymatic racemate resolution

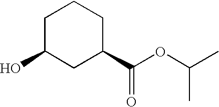

At room temperature, 100 g of racemic isopropyl 3-hydroxycyclohexane-1-carboxylate were stirred slowly with 200 ml of vinyl acetate, 800 ml of methylene chloride and 20 g of Novozyme® 435. After about 60% conversion (GC), the mixture was filtered off and concentrated under reduced pressure. This gave 110 g of product which were chromatographed on 1 kg of silica gel (n-heptane/EA 2:1): 1. Fraction, 70.9 g, isopropyl (1S,3R)-3-acetoxycyclohexane-1-carboxylate; ¹H-NMR (CDCl₃): □=1.22 (d, 6 H), 1.2-1.6 (m, 4 H), 1.8-2.0 (m, 3 H), 2.03 (s, 3 H), 2.20 (m, 1 H), 2.36 (m, 1 H), 4.70 (m, 1 H), 4.99 (sept, 1 H); 62% ee (HPLC on Chiralpak ADH 32 250×4.6; 1 ml/min, heptane/EtOH 3:1). 2. Fraction, 35.9 g, isopropyl (1R,3S)-3-hydroxycyclohexane-1-carboxylate; ¹H-NMR (CDCl₃): □=1.23 (d, 6 H), 1.20-1.45 (m, 4 H), 1.68 (d, 1 H), 1.86 (m, 2 H), 1.95 (m, 1 H), 2.18 (m, 1 H), 2.34 (m, 1 H), 3.63 (m, 1 H), 5.00 (sept, 1 H); ); >99% ee (HPLC on Chiralpak ADH 32 250×4.6; 1 ml/min, heptane/EtOH 3:1).

### Example 10

### Preparation of (1R,3S)-3-hydroxycyclohexane-1-carboxylic acid by basic hydrolysis of optically pure isopropyl (1R,3S)-3-hydroxycyclohexane-1-carboxylate

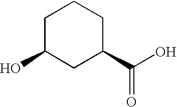

1 g (5.4 mmol) of isopropyl (1R,3S)-3-hydroxycyclohexane-1-carboxylate was stirred at RT in 6 ml of 2N NaOH overnight. A few drops of 11N NaOH were added, and the mixture was again stirred overnight. No more ester could be detected. The mixture was acidified with HCl and concentrated under reduced pressure, the residue was digested with isopropanol and filtered and the resulting solution was concentrated under reduced pressure; yield: 0.6 g; ¹H-NMR (D₂O) agrees with the NMR data of the racemic acid.

### Example 11

### Preparation of tert-butyl 2(S)-[((1R,3S)-3-hydroxycyclohexanecarbonyl)amino]-3-methylbutyrate by coupling (1R,3S)-3-hydroxycyclohexane-1-carboxylic acid with tert-butyl (S)-valinate hydrochloride

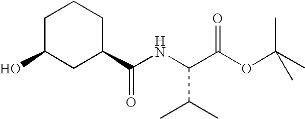

8.15 g (56.5 mmol) of (1R,3S)-3-hydroxycyclohexane-1-carboxylic acid, 19.6 ml of triethylamine and 11.86 g (56.5 mmol) of tert-butyl (S)-valinate hydrochloride were initially charged in 100 ml of DMF and cooled to 0° C., and 22.2 g (67.8 mmol) of TOTU ([cyano(ethoxycarbonyl)methyleneamino]-1,1,3,3-tetramethyluronium tetrafluoroborate) were added a little at a time. The reaction solution was stirred at 18-22° C. overnight. According to LC-MS, the starting material had been converted completely. The DMF was evaporated under reduced pressure and the residue was taken up in ethyl acetate and washed with aqueous NaHCO₃. The organic phase was dried (MgSO₄) and concentrated under reduced pressure: this gave 29.4 g of a red-brown oil. For purification, the product was chromatographed on silica gel (n-heptane/EA 2/1-1/1): this gave 11.8 g of a yellow solid which was directly reacted further; ¹H-NMR (CDCl₃): □=0.9 (m, 6 H), 1.25-1.57 (m, 4 H), 1.47 (s, 9 H), 1.78-1.95 (m, 3 H), 2.07-2.35 (m, 3 H), 3.67 (m, 1 H), 4.45 (m, 1 H), 6.0 (d, br., 1 H).

By alkylation with 4-iodomethyl-5-methyl-2-p-tolyloxazole, tert-butyl 2(S)-[((1R,3S)-3-hydroxycyclohexanecarbonyl)amino]-3-methylbutyrate can be converted into tert-butyl 3-methyl-2(S)-{[(1R,3S)-3-(5-methyl-2-p-tolyloxazol-4-yl methoxy)cyclohexane-carbonyl]amino}butyrate.

### Example 12

### Preparation of (1S,3R)-3-hydroxycyclohexane-1-carboxylic acid from isopropyl (1S,3R)-3-acetoxycyclohexane-1-carboxylate by enzymatic ester hydrolysis

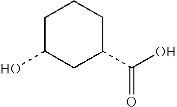

At RT, 10 g of isopropyl (1S,3R)-3-acetoxycyclohexane-1-carboxylate were stirred in 100 ml of phosphate buffer, pH=7, with 5 g of Novozyme 435 for 20 h. No more ester could be detected (TLC or GC). The immobilized enzyme was filtered off and the filtrate was acidified with HCl and concentrated under reduced pressure. The residue was digested with isopropanol. After filtration, the resulting clear solution was concentrated under reduced pressure; yield:

7.2 g; ¹H-NMR (D₂O) agrees with the NMR data of the racemic acid.

### Example 13

### Preparation of tert-butyl 2(R)-[((1S,3R)-3-hydroxycyclohexanecarbonyl)amino]-3-methylbutyrate by coupling (1S,3R)-3-hydroxycyclohexane-1-carboxylic acid with tert-butyl (R)-valinate hydrochloride

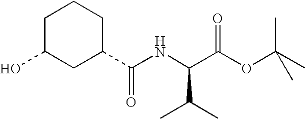

2.78 g (19.3 mmol) of (1S,3R)-3-hydroxycyclohexane-1-carboxylic acid, 6.7 ml of triethylamine and 4.04 g (19.3 mmol) of tert-butyl (R)-valinate hydrochloride were initially charged in 60 ml of DMF and cooled to 0° C., and 7.57 g (23.1 mmol) of TOTU ([cyano(ethoxycarbonyl)methyleneamino]-1,1,3,3-tetramethyluronium tetrafluoroborate) were added a little at a time. The reaction solution was stirred at 18-22° C. overnight. The DMF was evaporated under reduced pressure and the residue was taken up in ethyl acetate and washed with aqueous NaHCO₃. The organic phase was dried (MgSO₄), concentrated under reduced pressure and, for purification, chromatographed on silica gel (n-heptane/EA 2/1-1/1): this gave 4.96 g of a yellow solid; ¹H-NMR (CDCl₃): □=0.9 (m, 6 H), 1.25-1.57 (m, 4 H), 1.47 (s, 9 H), 1.78-1.95 (m, 3 H), 2.07-2.35 (m, 3 H), 3.67 (m, 1 H), 4.45 (m, 1 H), 6.0 (d, br., 1 H).

By alkylation with 4-iodomethyl-5-methyl-2-p-tolyloxazole, tert-butyl 2(R)-[((1S,3R)-3-hydroxycyclohexanecarbonyl)amino]-3-methylbutyrate can be converted into tert-butyl 3-methyl-2(R)-{[(1S,3R)-3-(5-methyl-2-p-tolyloxazol-4-ylmethoxy)cyclohexane-carbonyl]amino}butyrate.

For scheme C:

### Example 14

### Preparation of racemic cis-3-hydroxycyclohexane-1-carboxylic acid by basic hydrolysis of rac-6-oxabicyclo[3.2.1]octan-7-one

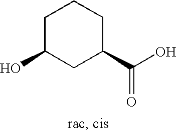

1 g (7.9 mmol) of rac-6-oxabicyclo[3.2.1]octan-7-one and 0.64 g (2 eq.) of NaOH in 20 ml of water were stirred at RT overnight. The mixture was acidified with HCl or acetic acid and concentrated under reduced pressure, the residue was digested with ethyl acetate or isopropanol and the resulting organic solution was concentrated under reduced pressure; yield: 0.6-0.7 g; ¹H-NMR (D₂O): □=0.9-1.2 (m, 4 H), 1.6-1.77 (m, 3 H), 1.95 (m, 1 H), 2.17 (m, 1 H), 3.45 (m, 1 H).

### Example 15

### Preparation of racemic methyl cis-3-hydroxycyclohexane-1-carboxylate

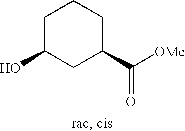

7 ml of acetyl chloride were added slowly, with stirring and gentle cooling with ice, to 10 g (79.3 mmol) of rac-6-oxabicyclo[3.2.1]octan-7-one and 100 ml of methanol. During the addition, the temperature increased to 45° C. but, within 10 min fell to 30° C. After 1 h, the reaction had ended and the reaction mixture was concentrated under reduced pressure; yield: 12.1 g; ¹H-NMR (CDCl₃): □=1.15-1.50 (m, 5 H), 1.8-2.0 (m, 3 H), 2.2 (m, 1 H), 2.37 (m, 1 H), 3.65 (m, 1 H), 3.7 (s, 3 H).

### Example 16

### Preparation of racemic cis-3-hydroxycyclohexane-1-carboxylic acid by basic hydrolysis of the racemic methyl ester

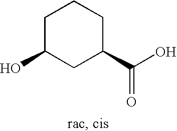

1 g (6.3 mmol) of methyl cis-3-hydroxycyclohexane-1-carboxylate was stirred at RT in 5 ml of THF, 1 ml of water and 1 ml of 11N NaOH overnight. No more ester could be detected. The mixture was acidified with HCl and concentrated under reduced pressure, the residue was digested with ethyl acetate and filtered and the solution was concentrated under reduced pressure; yield:

0.7g; ¹H-NMR (D₂O) agrees with the NMR data of the previous example.

### Example 17

### Preparation of racemic benzyl cis-3-hydroxycyclohexane-1-carboxylate

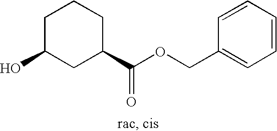

0.99 g (7.9 mmol) of rac-6-oxabicyclo[3.2.1]octan-7-one in 4 ml of DMF was stirred at 20-23° C. with 0.97 ml (1.2 eq.) of benzyl alcohol and 1.3 g (2.2 eq.) of potassium carbonate. After the reaction had ended, water was added and the mixture was extracted with MTBE. The combined organic phases were washed with saturated NaCl solution, dried with MgSO₄ and concentrated under reduced pressure.

Chromatography on silica gel (CH₂Cl₂—CH₂Cl₂/acetone 19:1-CH₂Cl₂/MeOH 18:1) gave 0.55 g of the desired product; ¹H-NMR (CDCl₃) agrees with the ¹H-NMRfrom the reaction of racemic cis-3-hydroxycyclohexane-1-carboxylic acid cesium salt with benzyl bromide.

### Example 18

### Preparation of cis-3-hydroxycyclohexane-1-carboxylic acid by de-hydrogenation of benzyl cis-3-hydroxycyclohexane-1-carboxylate

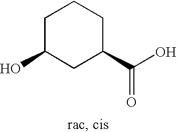

4 g (17.1 mmol) of benzyl cis-3-hydroxycyclohexane-1-carboxylate were dissolved at RT in 100 ml of MeOH, a catalytic amount of Pd (10% on carbon) was added and the mixture was hydrogenated at 5 bar. After the reaction had ended (TLC, LCMS), the catalyst was filtered off through Celite(R) and the solvent was evaporated under reduced pressure. The turbid residue was digested with ethyl acetate and filtered, and the resulting solution was concentrated under reduced pressure; yield: 2.0 g; ¹H-NMR (D₂O) agrees with the NMR data mentioned above.

### Example 19

### Preparation of tert-butyl 2(S)-[((1R,3S)-3-hydroxycyclohexanecarbonyl)amino]-3-methylbutyrate by coupling racemic cis-3-hydroxycyclohexane-1-carboxylic acid with tert-butyl (S)-valinate hydrochloride and subsequent stereoselective enzymatic acylation

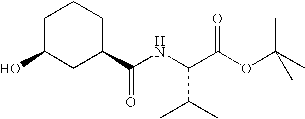

Racemic cis-3-hydroxycyclohexane-1-carboxylic acid and optically pure tert-butyl (S)-valinate hydrochloride were coupled in the presence of triethylamine in DMF using TOTU ([cyano(ethoxycarbonyl)methyleneamino]-1,1,3,3-tetramethyluronium tetrafluoroborate) (for the reaction conditions, see example 11), and the reaction solution was worked up.

10 g of the diastereomer mixture obtained in this manner in 300 ml of vinyl acetate were stirred at 20-23° C. with 1 g of lipase B from Candida Antarctica. After about 53% conversion, the stereoselective acylation of the hydroxyl group was terminated by filtering off the enzyme. Chromatography gave 4 g of tert-butyl 2(S)-[((1R,3S)-3-hydroxycyclohexanecarbonyl)amino]-3-methylbutyrate with 96.4% de (HPLC on Chiralpak (R) AD 250×4.6; 1 ml/min, heptane/EtOH/MeOH 20:1:1+0.1% TFA) and

6 g of the (S)-2-(1S,3R)-acetate with >80% de.

By alkylation with 4-iodomethyl-5-methyl-2-p-tolyloxazole, the substance was converted into tert-butyl 3-methyl-2(S)-{[(1R,3S)-3-(5-methyl-2-p-tolyloxazol-4-ylmethoxy)cyclohexanecarbonyl]amino}butyrate.

For scheme D:

### Example 20

### Synthesis of isopropyl (1R,3S)-3-(tert-butyldiphenylsilanyloxy)cyclohexane-carboxylate

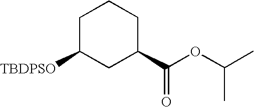

5.8 g of optically pure isopropyl (1R,3S)-3-hydroxycyclohexane-1-carboxylate were dissolved in 20 ml of DMF, and 9.74 ml of TBDPSCI (tert-butyldiphenylsilyl chloride), 3.2 g of imidazole and 100 mg of DMAP (dimethylaminopyridine) were added and the mixture was stirred at 18-23° C. for 4 h. Most of the solvent was distilled off under reduced pressure and the oily residue was partitioned between MTBE and water. The organic phase was dried (MgSO₄) and the solvent was then distilled off under reduced pressure; yield: 14 g of crude product. Chromatography on silica gel (EA/n-heptane 1:6) gave

7.0 g of isopropyl (1R,3S)-3-(tert-butyldiphenylsilanyloxy)cyclohexanecarb-oxylate; ¹H-NMR (CDCl₃): ||=1.04 (s, 9 H), 1.19 (d, 6 H), 1.0-1.35 (m, 3 H), 1.48 (m, 1 H), 1.65-1.82 (m, 3 H), 2.06 (m, 2 H), 3.57 (m, 1 H), 4.95 (m, 1 H), 7.34-7.43 (m, 6 H), 7.66-7.68 (m, 4 H).

### Example 21

### Synthesis of methyl (1R,3S)-3-(tert-butyldiphenylsilanyloxy)cyclohexanecarboxylate

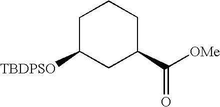

11.5 g of methyl (1R,3S)-3-hydroxycyclohexane-1-carboxylate with >99% ee were dissolved in 100 ml of DMF, 21.5 g of TBDPSCI (tert-butyldiphenylsilyl chloride), 6.5 g of imidazole and 500 mg of DMAP (dimethylaminopyridine) were added and the mixture was stirred at 18-23° C. overnight. Most of the solvent was distilled off under reduced pressure and the oily residue was taken up in MTBE and washed with water. The organic phase was dried (MgSO₄) and the solvent was then distilled off under reduced pressure. This gave 28 g of methyl (1R,3S)-3-(tert-butyldiphenylsilanyloxy)cyclohexanecarboxylate as a yellowish oil; C₂₄H₃₂O₃Si (396.61), MS (ESI): 397 (M+H⁺).

The substance can be used directly for the basic ester hydrolysis and the subsequent coupling with amino acid derivatives such as, for example, tert-butyl alaninate, leucinate and valinate, or the corresponding hydrochloride.

### Example 22

### Two-step synthesis of tert-butyl 2(S)-{[(1R,3S)-3-hydroxycyclohexanecarbonyl]-amino}-(3S)-methylbutyrate from (1R,3S)-3-(tert-butyldiphenylsilanyloxy)-cyclohexane-1-carboxylic acid

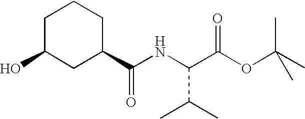

With stirring, 0.35 g of optically pure (1R,3S)-3-(tert-butyldiphenylsilanyloxy)-cyclohexanecarboxylic acid (prepared by hydrolysis of, for example, the corresponding isopropyl ester with NaOH in water/isopropanol), 0.38 g of t-butyl (S)-valinate hydrochloride and 0.45 ml of triethylamine were initially charged in 4 ml of DMF. At 0-5° C., 0.36 g of TOTU ([cyano(ethoxycarbonyl)methyleneamino]-1,1,3,3-tetramethyluronium tetrafluoroborate) was added in small portions, and, with cooling, the mixture was stirred for about 5 minutes. Stirring was then continued at RT. After 16 h, the reaction had ended. The entire reaction mixture was poured into about 60 ml of water and extracted twice with in each case 50 ml of ethyl acetate, and the organic phase was dried with MgSO₄ and concentrated under reduced pressure. The residue (1.4 g) was purified on silica gel (n-heptane/EA 1:1); yield: 394 mg of a white solid.

To remove the TBDPS protective group, tert-butyl 2(S)-{[(1R,3S)-3-(tert-butyldiphenylsilanyloxy)cyclohexanecarbonyl]amino}-3-methylbutyrate was reacted in THF with tetrabutylammonium fluoride. Concentration of the reaction mixture and purification on silica gel (n-heptane/EA 3:1) gave 197 mg of the desired compound; C₁₆H₂₉NO₄ (299.41), MS (ESI): 300 (M+H⁺); HPLC (Chiralpak AD 250×4.6; 1 ml/min, heptane/EtOH/MeOH 20:1:1+0.1% TFA): Rt=4.9 min.

For schemes A, B and D

### Example 23

### Preparation of methyl (1S,3R)-3-hydroxycyclohexane-1-carboxylate from isopropyl (1S,3R)-3-acetoxycyclohexane-1-carboxylate—via (1S,5R)-6-oxabicyclo[3.2.1]octan-7-one

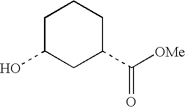

2.74 g of isopropyl (1S,3R)-3-acetoxycyclohexane-1-carboxylate from the stereoselective enzymatic ester formation of racemic isopropyl cis-3-hydroxycyclo-hexane-1-carboxylate in vinyl acetate/methylene chloride using Novozyme 435 were, at 20-23° C., converted in virtually quantitative yield into (1S,5R)-6-oxabicyclo[3.2.1]-octan-7-one by stirring with 0.3 g of K₂CO₃ in 30 ml of methanol. After filtration and concentration of the solvent under reduced pressure, (1R,5S)-6-oxabicyclo[3.2.1]-octan-7-one

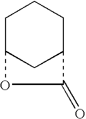

was taken up in about 30 ml of methanol and, with stirring and gentle cooling with ice, 1-2 ml of acetyl chloride were added. After the reaction had ended, the reaction mixture was concentrated under reduced pressure; yield: 1.5 g; ¹H-NMR (CDCl₃): □=1.15-1.50 (m, 5 H), 1.8-2.0 (m, 3 H), 2.2 (m, 1 H), 2.37 (m, 1 H), 3.65 (m, 1 H), 3.7 (s, 3 H).

Methyl (1S,3R)-3-hydroxycyclohexane-1-carboxylate was directly reacted further.

For schemes A, B, C, D, E—two-step conversion of the compound of the formula (II) into a racemic compound of the formula (IV)

### Example 24

### Preparation of the racemic cis-3-hydroxycyclohexane-1-carboxylic acid cesium salt

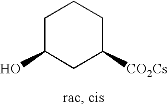

10 g (79.3 mmol) of rac-6-oxabicyclo[3.2.1]octan-7-one and 13.5 g (80.3 mmol) of cesium hydroxide monohydrate were stirred in 50 ml of water at RT. After 2 h, the reaction had ended. The mixture was concentrated under reduced pressure, and twice, in each case 50 ml of DMF were added and the mixture was concentrated under reduced pressure; yield: 19.6 g (89.5%); ¹H-NMR (D₂O): □=1.1-1.37 (m, 4 H), 1.75-2.25 (m, 5 H), 3.63 (m, 1 H).

### Example 25

### Preparation of racemic benzyl cis-3-hydroxycyclohexane-1-carboxylate

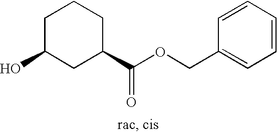

2 g (7.24 mmol) of racemic cis-3-hydroxycyclohexane-1-carboxylic acid cesium salt and 1.1 g (0.81 ml, 6.82 mmol) of benzyl bromide were stirred in 10 ml of DMF at RT. After 4 hours of stirring and standing overnight, the benzylation was complete. The reaction mixture was put into about 100 ml of water and extracted twice with in each case about 50 ml of MTBE. The combined organic phases were washed once with water and then dried using MgSO₄ and concentrated under reduced pressure; crude yield: 1.3 g (76.6%). The crude product was either directly reacted further or chromatographed; ¹H-NMR (CDCl₃): □=1.17-1.50 (m, 4 H), 1.62 (d, 1 H), 1.80-2.0 (m, 3 H), 2.22 (m, 1 H), 2.43 (m, 1 H), 3.62 (m, 1 H), 5.12 (s, 2 H), 7.28-7.40 (m, 5 H).

Further examples of the enzymatic racemate resolution by stereoselective ester formation

### Example 26

### Stereoselective acylation of methyl cis-3-hydroxycyclohexane-1-carboxylate

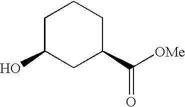

500 mg of racemic methyl 3-hydroxycyclohexane-1-carboxylate, 1 ml of vinyl acetate, 4 ml of methylene chloride and 25 mg of Novozyme 435 were stirred slowly at room temperature. After about 54% conversion (GC), the reaction was terminated by filtering off the enzyme. The optical purity of methyl (1R,3S)-3-hydroxycyclo-hexane-1-carboxylate was determined as being >99% ee.

### Example 27

### Synthesis of benzyl (1R,3S)-3-hydroxycyclohexane-1-carboxylate by stereoselective acylation of racemic benzyl cis-3-hydroxycyclohexane-1-carboxylate

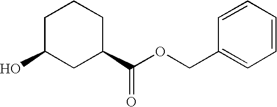

For 2 h, 108 mg (0.5 mmol) of racemic benzyl 3-hydroxycyclohexane-1-carboxylate were slowly stirred at room temperature in 5 ml of vinyl acetate and 4 ml of methylene chloride with 54 mg of Novozym 435. After filtration and concentration under reduced pressure, the product was chromatographed on silica gel (n-heptane/EA 2:1); yield: 56 mg of benzyl (1R,3S)-3-hydroxycyclohexane-1-carboxylate; ¹H-NMR (CDCl₃): □=1.15-1.5 (m, 4 H), 1.63 (d, 1 H), 1.85 (m, 1 H), 1.95 (m, 2 H), 2.23 (m, 1 H), 2.41 (m, 1 H), 3.62 (m, 1 H), 5.11 (s, 2 H), 7.35 (m, 5 H); ee>99% (HPLC on Chiracel OJ 250×4.6; 1 ml/min, heptane/EtOH/MeOH 70:1:1).

### Example 28

### Synthesis of benzyl (1R,3S)-3-hydroxycyclohexane-1-carboxylate by racemate resolution of racemic benzyl cis-3-hydroxycyclohexane-1-carboxylate

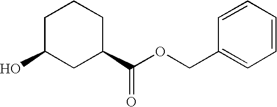

29.5 g of benzyl cis-(1RS,3SR)-3-hydroxycyclohexanecarboxylate were dissolved in about 200 ml of vinyl acetate, 15 g of Novozyme 435 were added and the mixture was stirred at 20-23° C. After 75 minutes, the enzyme was filtered off and the solution was concentrated under reduced pressure. Chromatography on silica gel (n-heptane/ethyl acetate 2:1) gave 12 g of benzyl (1R,3S)-3-hydroxycyclohexane-carboxylate; >99% ee (HPLC on Chiracel OJ 250×4.6; 1 ml/min, heptane/EtOH/CH₃OH 70:1:1); ¹H-NMR (DMSO), □=0.98-1.30 (m, 4 H), 1.66-1.82 (m, 4 H), 2.04 (m, 1 H), 2.39 (m, 1 H), 3.39 (m, 1 H), ), 4.63 (dd, 2 H), 5.08 (m, 1 H), 7.30-7.40 (m, 5 H).

The reaction also yielded 17 g of the (1S,3R)-acetyl compound; 94% ee (HPLC on Chiracel OJ 250×4.6; 1 ml/min, heptane/EtOH/CH₃OH 70:1:1, after removal of the acetyl group).

### Example 29

### Racemate resolution of isopropyl cis-3-hydroxycyclohexane-1-carboxylate

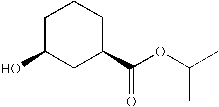

200 mg (1.07 mmol) of racemic isopropyl cis-3-hydroxycyclohexane-1-carboxylate were dissolved in 10 ml of acetone, 584 mg (5.78 mmol) of succinic anhydride and 40 mg of Novozyme 435 were added and the mixture was stirred at 5° C. After 40-45% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was 72% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was >97% ee (HPLC on Chiralpak AD-H 250×4.6; 1 ml/min, heptane/MeOH/EtOH/TFA 500:100:100:0.7).

### Example 30

### Racemate resolution of isopropyl cis-3-hydroxycyclohexane-1-carboxylate

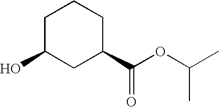

200 mg (1.07 mmol) of racemic isopropyl cis-3-hydroxycyclohexane-1-carboxylate were dissolved in 10 ml of DIPE, 584 mg (5.78 mmol) of succinic anhydride and 40 mg of Novozym 435 were added and the mixture was stirred at 35° C. After about

40% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was 61% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was 94% ee (HPLC on Chiralpak AD-H 250×4.6; 1 ml/min, heptane/MeOH/EtOH/TFA 500:100:100:0.7).

### Example 31

### Racemate resolution of isopropyl cis-3-hydroxycyclohexane-1-carboxylate

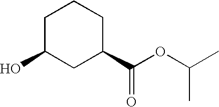

200 mg (1.07 mmol) of racemic isopropyl cis-3-hydroxycyclohexane-1-carboxylate were dissolved in 10 ml of acetone, 584 mg (5.78 mmol) of succinic anhydride and 160 mg of Novozym 435 were added and the mixture was stirred at 35° C. After 45-49% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was 84% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was 96% ee (HPLC on Chiralpak AD-H 250×4.6; 1 ml/min, heptane/MeOH/EtOH/TFA 500:100:100:0.7).

### Example 32

### Racemate resolution of isopropyl cis-3-hydroxycyclohexane-1-carboxylate

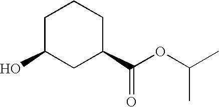

200 mg (1.07 mmol) of racemic isopropyl cis-3-hydroxycyclohexane-1-carboxylate were dissolved in 10 ml of MTB ether, 119 mg (1.18 mmol) of succinic anhydride and 160 mg of Novozym 435 were added and the mixture was stirred at 35° C. After 33-37% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was 46% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was 93% ee (HPLC on Chiralpak AD-H 250×4.6; 1 ml/min, heptane/MeOH/EtOH/TFA 500:100:100:0.7).

### Example 33

### Racemate resolution of isopropyl cis-3-hydroxycyclohexane-1-carboxylate

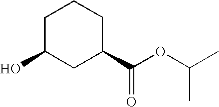

200 mg (1.07 mmol) of racemic isopropyl cis-3-hydroxycyclohexane-1-carboxylate were dissolved in 10 ml of THF, Novozym 435 L-2 was added and the mixture was stirred at 35° C. After about 40% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was 64% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was 97% ee (HPLC on Chiralpak AD-H 250×4.6; 1 ml/m in, heptane/MeOH/EtOH/TFA 500:100:100:0.7).

### Example 34

### Racemate resolution of isopropyl cis-3-hydroxycyclohexane-1-carboxylate

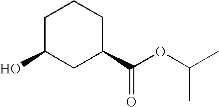

200 mg (1.07 mmol) of racemic isopropyl cis-3-hydroxycyclohexane-1-carboxylate were dissolved in 10 ml of DIPE, 584 mg (5.78 mmol) of succinic anhydride and 40 mg of Novozym 435 were added and the mixture was stirred at 35° C. After 40-45% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was 70% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was 92% ee (HPLC on Chiralpak AD-H 250×4.6; 1 ml/min, heptane/MeOH/EtOH/TFA 500:100:100:0.7).

### Example 35

### Racemate resolution of isopropyl cis-3-hydroxycyclohexane-1-carboxylate

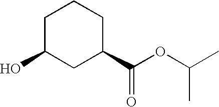

200 mg (1.07 mmol) of racemic isopropyl cis-3-hydroxycyclohexane-1-carboxylate were dissolved in 10 ml of n-heptane, 119 mg (1.18 mmol) of succinic anhydride and 160 mg of Novozym 435 were added and the mixture was stirred at 5° C. At about 30% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was 43% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was 96% ee (HPLC on Chiralpak AD-H 250×4.6; 1 ml/min, heptane/MeOH/EtOH/TFA 500:100:100:0.7).

### Example 36

### Racemate resolution of isopropyl cis-3-hydroxycyclohexane-1-carboxylate

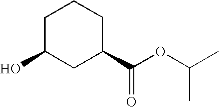

200 mg (1.07 mmol) of racemic isopropyl cis-3-hydroxycyclohexane-1-carboxylate were dissolved in 10 ml of toluene, 584 mg (5.78 mmol) of succinic anhydride and 160 mg of Novozym 435 were added and the mixture was stirred at 5° C. At about

46-49% conversion, the reaction was terminated by filtering off the enzyme. Using a concentrated sample, the optical purities both of the un-reacted substrate and of the acylation product formed were determined. The optical purity of isopropyl (1R,3S)-3-hydroxycyclohexane-1-carboxylate was >76% ee (HPLC on Chiralpak AD-H 250×4.6; 1 ml/min, heptane/MeOH/EtOH/TFA 500:100:100:0.7), the optical purity of mono(cis-3-isopropoxycarbonylcyclohexyl) (1R,3S)-succinate was 91% ee (HPLC on Chiralpak AD-H 250×4.6; 1 ml/min, heptane/MeOH/EtOH/TFA 500:100:100:0.7).

### Example 37

### Preparation of isopropyl (1R,3S)-3-hydroxycyclohexane-1-carboxylate and mono(-3-isopropoxycarbonylcyclohexyl) (1R,3S)-succinate by stereoselective enzymatic acylation of isopropyl cis-3-hydroxycyclohexane-1-carboxylate with succinic anhydride

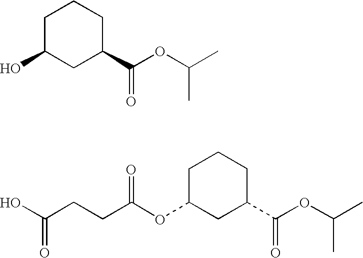

2.0 mg (10.7 mmol) of racemic isopropyl cis-3-hydroxycyclohexane-1-carboxylate were dissolved in 100 ml of MTB ether, 5.84 g (57.8 mmol) of succinic anhydride and 1.6 g of Novozym 435 were added and the mixture was stirred at 35° C. At about 50% conversion, the reaction was terminated by filtering off the enzyme. The reaction mixture was dissolved in acetone, concentrated under reduced pressure and then partitioned between diisopropyl ether/n-heptane 4:1 and 1M Na₂CO₃ (aq.). Concentration of the organic phase gave 890 mg of isopropyl (1R,3S)-3-hydroxy-cyclohexane-1-carboxylate; ¹H-NMR (CDCl₃): □=1.23 (d, 6 H), 1.20-1.45 (m, 4 H), 1.68 (d, 1 H), 1.86 (m, 2 H), 1.95 (m, 1 H), 2.18 (m, 1 H), 2.34 (m, 1 H), 3.63 (m, 1 H), 5.00 (sept, 1 H); optical purity: 90% ee (HPLC on Chiralpak AD-H 250×4.6; 1 ml/min, heptane/MeOH/EtOH/TFA 500:100:100:0.7).

Using 2N HCl, the aqueous phase was adjusted to pH 6-7 and extracted twice with ethyl acetate. Concentration after drying (Na₂SO₄) gave 1.39 g of mono(3-isopropoxycarbonylcyclohexyl) (1R,3S)-succinate; ¹H-NMR (CDCl₃): □=1.22 (d, 6 H), 1.24-1.41 (m, 3H), 1.47 (q, 1 H), 1.83-2.01 (m, 3 H), 2.17-2.24 (m, 1H), 2.35 (tt, 1 H), 2.58-2.64 (m, 2 H), 2.64-2.71 (m, 2 H), 4.69-4.79 (m, 1 H), 4.99 (sept, 1 H); optical purity: 88% ee (HPLC on Chiralpak AD-H 250×4.6; 1 ml/min, heptane/MeOH/EtOH/TFA 500:100:100:0.7).

Isolated products or crude product mixtures were identified by ¹H-NMR and/or mass spectra and/or by GC or HPLC.

The optical purity of the esters and alcohols was determined by HPLC, for example on Chiralpak AD 250×4.6 (Daicel) or Chiracel OD 250×4.6.

What is claimed is:

1. A process for the preparation of chiral, non-racemic compounds of the formulae (Ia) and (Ib) 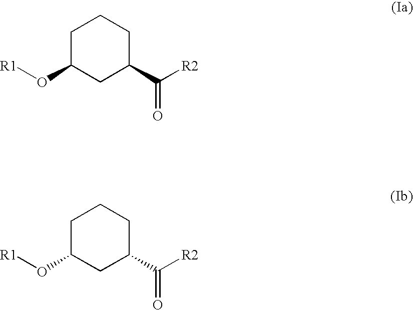
wherein:
R1 is 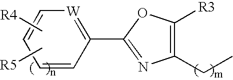
in which:
R3 is selected from the group consisting of H, (C1-C6)-alkyl, (C3-C8)-cycloalkyl, (C1-C3)-alkyl-(C3-C8)-cycloalkyl, phenyl, (C1-C3)-alkyl-phenyl, (C5-C6)-heteroaryl, (C1-C3)-alkyl-(C5-C6)-heteroaryl or (C1-C3)-alkyl which may be fully or partially substituted by F;
R4 and R5 are independently selected from the group consisting of H, F, Cl, Br, CF₃, OCF₃, (C1-C6)-alkyl, O—(C1-C6)-alkyl, SCF₃, SF₅, OCF₂—CHF₂, (C6-C10)-aryl, (C6-C10)-aryloxy, OH, NO₂; or,
R4 and R5 together with the carbon atoms that carry them form a fused partially saturated or unsaturated bicyclic (C6-C10)-aryl or (C5-C11)-heteroaryl ring;
W is CH or N, if n=1;
W is O, S or NR⁶, if n=0;
m is a whole integer from 1-6;
R6 is selected from the group consisting of H, (C1-C6)-alkyl-phenyl, (C1-C6)-alkyl; or,
is an OH protective group (PG) is selected from the group consisting of benzyloxymethyl, benzyl, para-methoxybenzyl, tert-butyldimethylsilyl (TBDMS), tert-butyldiphenylsilyl (TBDPS), tetrahydropyranyl (THP), 1-ethoxyethyl (EE), 1-methyl-1-methoxyethyl or 1-methyl-1-benzyloxyethyl; and,
R2 is selected from the group consisting of: 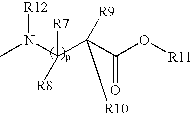
wherein:
p is an integer from 0-2;
R7 is H, (C1-C6)-alkyl;
R8 is H, (C1-C6)-alkyl;
R9 is H, F, (C1-C6)-alkyl;
R10 is selected from the group consisting of H, F, (C1-C6)-alkyl, O—(C1-C6)-alkyl, (C2-C6)-alkenyl, (C2-C6)-alkynyl, (C3-C8)-cycloalkyl, phenyl, wherein the alkyl, alkenyl, alkynyl and cyclically groups may optionally be substituted by one or more moieties from the group consisting of hydroxyl, phenyl, (C5-C11)-heteroaryl, O—(C1-C6)-alkyl and NR13R14, and phenyl which also may optionally be substituted by one or more groups consisting of hydroxyl, O—(C1-C6)-alkyl, F and CF₃, with the proviso that R10 is not NR13R14 or O—(C1-C6)-alkyl, if R9=F;
R8 and R10 when fused together with the carbon atom that carries them are (C3-C8)-cycloalkyl;
R10 and R12 when fused together are pyrrolidine and piperidine, if n=0;
R11 is selected from the group consisting of H, (C1-C8)-alkyl, benzyl, (C1-C4)-alkyl-(C6-C10)-aryl, (C1-C4)-alkyl-O—(C1-C4)-alkyl, phenyl-(C1-C4)-alkyl, where alkyl, benzyl, phenyl, aryl may optionally be mono- or polysubstituted by O—(C1-C6)-alkyl, OCH₂CH₂—OMe, F, Cl, Br, I, Si(CH₃)₃, OSi(CH₃)₃, Si(iPr)₃, OSi(iPr)₃, OCH₂CH₂—SiMe₃, OCH₂—Si(iPr)₃, O—CH₂—C₆H₅, SO₂C₆H₄-p-Me, SMe, CN, NO₂, CH₂COC₆H₅;
R12 is selected from the group consisting of H, (C1-C6)-alkyl, (C2-C6)-alkenyl, (C2-C6)-alkynyl, benzyl, CO—(C1-C6)-alkyl, CO-phenyl, C(O)—O—(C1-C6)-alkyl, allyloxycarbonyl (ALOC), benzyloxycarbonyl (Cbz, Z), 9-fluorenylmethyloxycarbonyl (FMOC), (C1-C4)-alkyl-(C6-C10)-aryl, (C1-C4)-alkyl-(C5-C11)-heteroaryl, (C1-C4)-alkyl-O—(C1-C4)-alkyl, phenyl-(C1-C4)-alkyl, (C5-C6)-heteroaryl-(C1-C4)-alkyl; SO₂-(C1-C6)-alkyl, SO₂-(C1-C6)-alkyl-SO₂-(C1-C6)-alkyl, SO₂-phenyl, wherein phenyl may optionally be substituted by (C1-C6)-alkyl, O—(C1-C6)-alkyl, F, Cl;
R13 is selected from the group consisting of (C1-C6)-alkyl;
R14 is selected from the group consisting of (C1-C6)-alkyl-phenyl, (C1-C6)-alkyl;
Said process comprising a reaction wherein
a) racemic 6-oxabicyclo[3.2.1]octan-7-one of the formula (II) 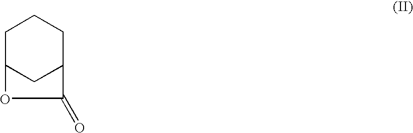
is reacted with a compound of the formula (III)
HO—R15 (III)
wherein
R15 is selected from the group consisting of H, (C1-C8)-alkyl, (C3-C8)-cycloalkyl, (C2-C8)-alkenyl, (C2-C8)-alkynyl, benzyl, (C1-C4)-alkyl-(C6-C10)-aryl, (C1-C4)-alkyl-(C5-C11)-heteroaryl, (C1-C4)-alkyl-O—(C1-C4)-alkyl, phenyl-(C1-C4)-alkyl, (C5-C6)-heteroaryl-(C1-C4)-alkyl, where alkyl, benzyl, phenyl, aryl, heteroaryl may optionally be mono- or polysubstituted by phenyl, O—(C1-C6)-alkyl, OCH₂CH₂—OMe, OTs, F, Cl, Br, I, Si(CH₃)₃, OSi(CH₃)₃, Si(iPr)₃, OSi(iPr)₃, OCH₂CH₂—SiMe₃, OCH₂—Si(iPr)₃, OTHP, O—CH₂—C₆H₅, SO₂C₆H₄-p-Me, SMe, CN, NO₂, COOH, CONH₂, CH₂COC₆H₅, CO-benzyloxy, CO—O(C1-C6)-alkyl, NHTs, NHAc, NHBoc, NHAloc, NHbenzyl;
in the presence of a suitable base or acid in a suitable solvent or with acid halides in alcohols, wherein the presence of water either the salt or the free acid is obtained to give a racemic cis-configured compound of the formula (IV), 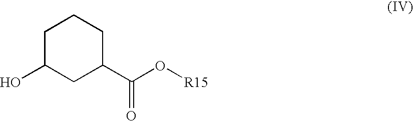
in which R15 is as defined above which optionally may be present in its' ionic form, as a Cs⁺, Li⁺, K⁺, NH₄⁺, Ca²⁺, Ba²⁺ or Mg2⁺ salt and in which R15 is also Cs, Li, K, NH₄, Ca, Ba, Mg, and optionally, the resulting product is further converted into another product of the formula (IV) followed by;
b) an enzymatic ester formation and compound separation whereby the resulting compounds of the formula (IV) exist as two different isomers which are subjected to a stereoselective enzymatic ester formation wherein an acyl donor and the enzyme are added to the hydroxyl compounds in an organic solvent and the resulting mixture is stirred at from −20 to 80° C. and, after the reaction has ended, one stereoisomer is present as an ester of formula (Vb) 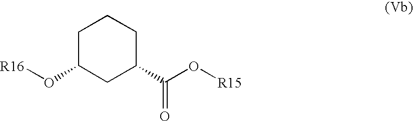
wherein:
R16 is selected from the group consisting of C(═O)—(C₁-C₁₆)-alkyl, C(═O)—(C₂-C₁₆)-alkenyl, C(═O)—(C₃-C₁₆)-alkynyl, C(═O)—(C₃-C₁₆)-cycloalkyl, wherein one or more carbon atoms may be substituted by oxygen atoms or by 1-3 moieties from the group consisting of F, Cl, Br, CF₃, CN, NO₂, hydroxyl, methoxy, ethoxy, phenyl, CO—O(C₁-C₄)-alkyl and CO—O(C₂-C₄)-alkenyl, where phenyl, CO—O(C₁-C₄)-alkyl and CO—O(C₂-C₄)-alkenyl for their part may be optionally substituted by 1-3 moieties selected from the group consisting of F, Cl, Br, CF₃, and
R15 is as defined above,
and the other stereoisomer is present unchanged as the alcohol of the formula (IVa) 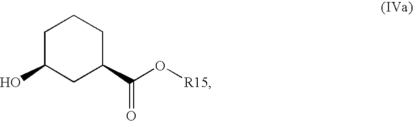
wherein said compounds are:
a) separated by extraction, chromatography or other methods utilizing their different chemical or physicochemical properties, or
b) processed further wherein the enantiomers of the formula (IVa) obtained as alcohols are processed by:
c) ester cleavage wherein the enantiomers of the formula (Vb) obtained as acylated compounds are hydrolyzed to give chemically enantiomeric alcohols (IVb), or,
d) by reaction with K₂CO₃ in methanol, trans-esterified intra-molecularly to give the optically active (1S,5R)-6-oxabicyclo[3.2.1]octan-7-one which is then converted into an isomeric form of the product (IVb): 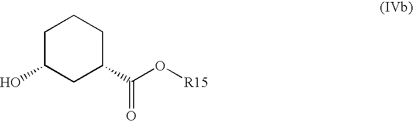
or
e) the compound of formula (Vb) is converted by lipase-catalyzed cleavage of both ester functions into the optically active compound of the formula (IVb) wherein R15 is hydrogen which can be converted into an isomeric form of the product.

2. A process for the preparation of chiral, non-racemic compounds of the formula (Ia) and (Ib) from compounds of the formula (VI)
R¹—X (VI)
in which
R¹ is 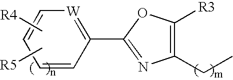
and R³, R⁴, R⁵, W, n and m are as defined above, or
R¹ is an OH protective group (PG) as defined above with the exception of THP, EE, 1-methyl-1-methoxyethyl or 1-methyl-1-benzyloxyethyl, and;
X is selected from the group consisting of Cl, Br, I, OTs, OMs, OTf;
in the presence of a base in a suitable solvent to yield compounds selected from the group consisting of formula (VIIa) or (VIIb); 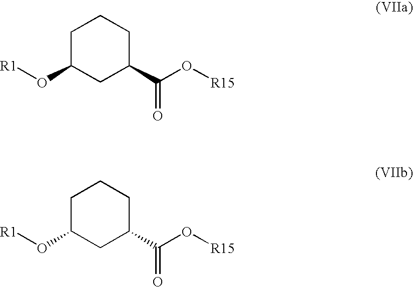
or,
wherein R1 is PG, compounds selected from the group consisting of formula (VIIIa) or (VIIIb) 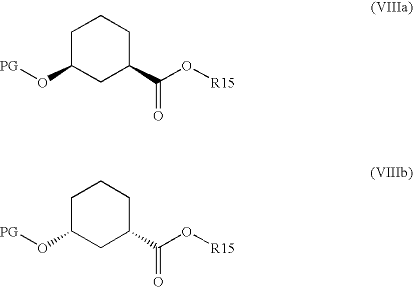
or
R¹ is an OH protective group (PG) selected from the group consisting of tetrahydropyranyl (THP), 1-ethoxyethyl, 1-methyl-1-methoxyethyl or 1-methyl-1-benzyloxyethyl; whereby compounds of formula (IVa) or (IVb) are reacted under acid catalysis with the appropriate known enol ethers, to yield compounds of the formula (VIIIa) or (VIIIb) followed by direct reaction (DR) or ester cleavage & coupling (EC & C).

3. A process for the preparation of chiral, non-racemic compounds of the formula (Ia) or (Ib) or the isomeric forms thereof, 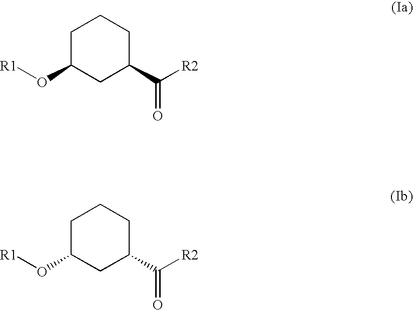
by direct conversion by reacting an amine of the formula (IX)
R2-H (IX)
in which
R2 is 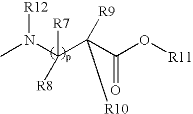
wherein R7, R8, R9, R10, R11, R12 and p are as defined above,
or the corresponding lithium or dimethylaluminum derivative thereof, or by reacting the compounds of the formula (VIIa), (VIIb), (VIIIa) or (VIIIb) or the amine or amino acid derivative R2-H of the formula (IX) in the presence of activating reagents or catalysts, to give compounds of the formula (Ia) or (Ib) or isomeric forms thereof, 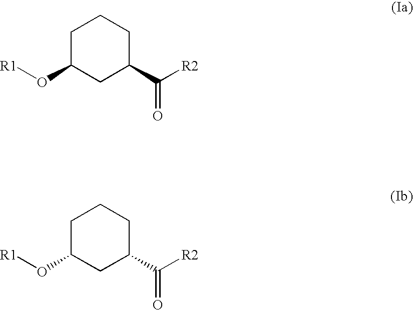
or, in the case of R1=PG, to yield compounds of the formula (Xa) or (Xb), 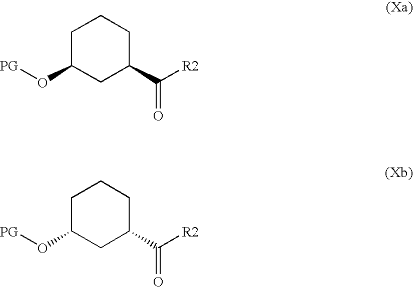
whereby compounds of formula (VIIa) or (VIIb) or (VIIIa) or (VIIIb) are subjected to an ester cleavage, and the resulting compounds of the formula (XIa), (XIb), (XIIIa) or (XIIIb) 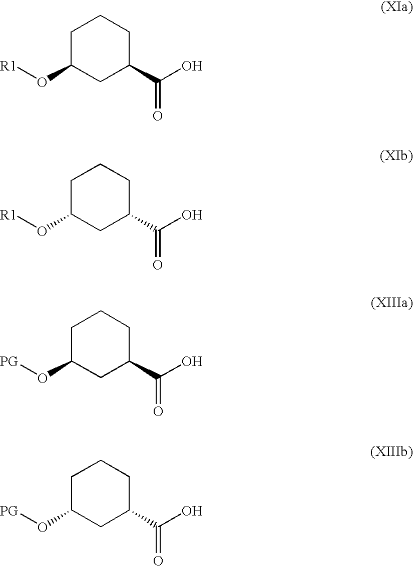
or the corresponding salt are subsequently coupled with a compound of the formula (IX)
R²—H (IX)
in which
R2 is 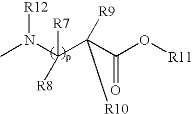
wherein R7, R8, R9, R10, R11, R12 and p are as defined above,
in the presence of dehydrating or activating reagents, to give a compound of the formula (Ia) or (Ib) or an isomeric form thereof; 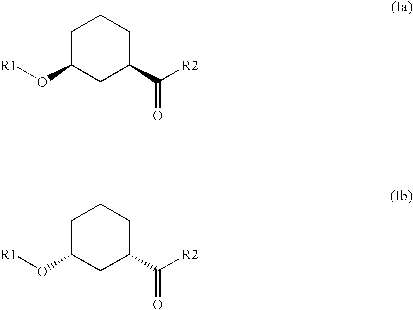
and, followed optionally if appropriate,
f) Removal of the protective group PG (RPG) wherein
if R¹ is an OH protective group (PG) as defined above under R¹, the compounds of the formula (Xa) or (Xb) 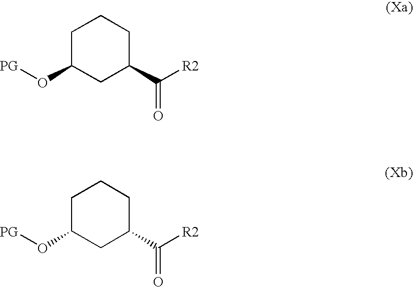
in which R² and PG are as defined above, are converted by removal of the protective group as is known in the art into compounds of the formula (XIIa) or (XIIb) 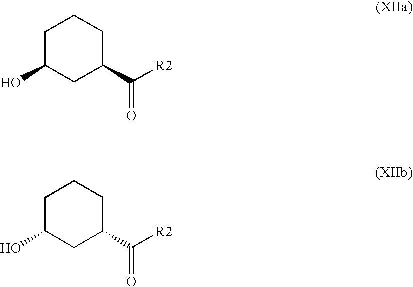
in which R² is as defined above, followed by conversion according to the stated process variants into the compounds of the formula (Ia) or (Ib) or isomeric forms thereof.

4. A process for the preparation of chiral, non-racemic compounds of the formulae (Ia) and (Ib) as recited in claim 1 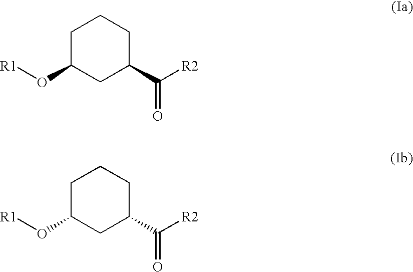
comprising a reaction scheme selected from the group consisting of:
A) LO→EF+S[→EC]→Alk-R¹→DR or EC+C→product compounds, or
B) LO→EF+S[→EC]→DR or EC+C→Alk-R¹→product compounds
or
C) LO→DR or EC+C→EF+S→[EC]→Alk-R¹→product compounds
or
D) LO→EF+S→[EC]→Alk-PG→DR or EC+C→RPG→Alk-R¹→product/isomeric form,
or
E) LO→Alk-PG→DR or EC+C→RPG→EF+S→[EC]→Alk-R¹→product/isomeric form.

5. A process for the preparation of chiral, non-racemic compounds of the formulae (Ia) and (Ib) as recited in claim 1 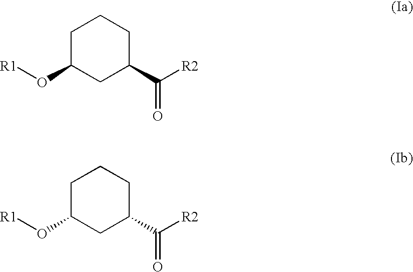
wherein the sequence of the individual reaction steps as described therein are as follows:
A) LO→EF+S[→EC]→Alk-R¹→DR or EC+C→product compounds, and
wherein all the recited substituents, equivalents and their definitions remain the same

6. A process for the preparation of chiral, non-racemic compounds of the formulae (Ia) and (Ib) as recited in claim 1 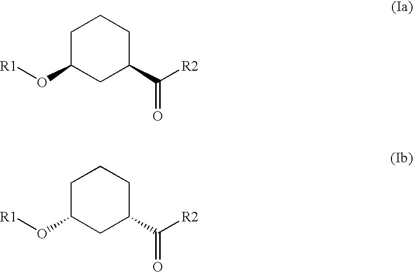
wherein the sequence of the individual reaction steps as described therein are as follows:
B) LO→EF+S[→EC]→DR or EC+C→Alk-R¹→product compounds
and
wherein all the recited substituents, equivalents and their definitions remain the same.

7. A process for the preparation of chiral, non-racemic compounds of the formulae (Ia) and (Ib) as recited in claim 1 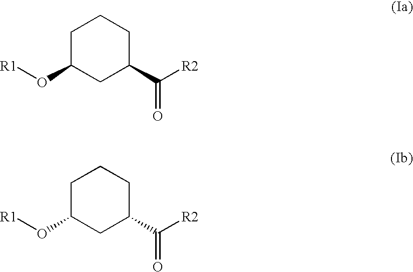
wherein the sequence of the individual reaction steps as described therein are as follows:
C) LO→DR or EC+C→EF+S→[EC]→Alk-R¹→product compounds
wherein all the recited substituents, equivalents and their definitions remain the same.

8. The process for the preparation of chiral, non-racemic compounds of the formula (Ia) and (Ib) as recited in claim 1 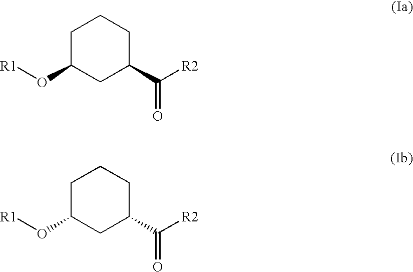
wherein the sequence of the individual reaction steps as described therein are as follows:
D) LO→EF+S→[EC]→Alk-PG→DR or EC+C→RPG→Alk-R¹→product compounds, and
wherein all the recited substituents, equivalents and their definitions remain the same.

9. The process for the preparation of chiral, non-racemic compounds of the formula (Ia) and (Ib) as recited in claim 1 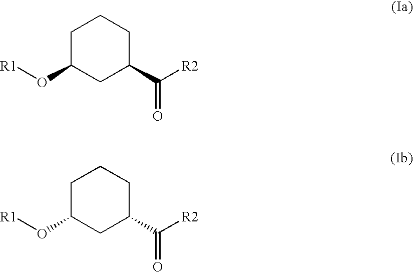
wherein the sequence of the individual reaction steps as described therein are as follows
E) LO→Alk-PG→DR or EC+C→RPG→EF+S→[EC]→Alk-R¹→product compounds and,
wherein all the recited substituents, equivalents and their definitions remain the same.

10. The process as recited in claim 3 wherein:
R4 is selected from the group consisting of Br, CF₃, OCF₃, (C1-C6)-alkyl, O—(C1-C6)-alkyl; and,
R5 is selected from the group consisting of H, (C1-C6)-alkyl, O—(C1-C6)-alkyl or
R4 and R5 together with the phenyl ring are a naphthyl group;
R3 is selected from the group consisting of CF₃, (C1-C6)-alkyl, (C3-C6)-cycloalkyl, phenyl;
W is CH, if n=1;
m is 1;
p is 0;
R9 is selected from the group consisting of H, (C1-C6)-alkyl;
R10 is selected from the group consisting of (C1-C6)-alkyl, wherein the alkyl may optionally be substituted by phenyl;
R10 and R12 together with the atoms that carry them are pyrrolidine, if p=0;
R9 and R10 together with the carbon atom that carries them are selected from the group consisting of (C3-C6)-cycloalkyl;
R11 is H, and
R12 is selected from the group consisting of H, (C1-C6)-alkyl or, benzyl.
